(19) United States
(12) Patent Application Publication
(10) Pub. No.: US 2011/0318272 A1
(43) Pub. Date: Dec. 29, 2011

(54) PESTICIDAL MIXTURES GIVING SYNERGISTIC PESTICIDAL EFFECTS

(75) Inventors: JOHN STREET, Aargau (CH); ALAN JUTSUM, Berkshire (GB)

(73) Assignee: ARYSTA LIFESCIENCE CORPORATION, Tokyo (JP)

(21) Appl. No.: 13/160,256

(22) Filed: Jun. 14, 2011

Related U.S. Application Data
(60) Provisional application No. 61/358,257, filed on Jun. 24, 2010.

(51) Int. Cl. A01N 65/12; A01P 7/02; A01P 7/04; A01P 7/00; A01N 65/00; A61K 49/00

(52) U.S. Cl. 424/9.2; 424/725; 424/764

(57) ABSTRACT

Compositions and methods are provided for synergistic pesticidal mixtures comprising a first pesticidal composition and a second pesticidal composition, wherein the pesticidal activity of the mixture exhibits synergistic effects compared with the expected effects of the mixtures based on the sum of the activity of the first pesticidal composition alone and the activity of the second pesticidal composition alone, wherein the pesticidal activity of the mixture allows the use of lower amounts of each component to achieve a desired level of control of the target pest, and/or provides a desired level of control of the target pest sooner after application of the mixture, compared with the amount and/or time required for first pesticidal composition alone, and the amount and/or time required for the second pesticidal composition alone, to achieve the same level of control. Synergistic pesticidal mixtures and methods according to the invention are provided comprising a first pesticidal composition comprising at least two essential oils, a carrier oil, and an emulsifier, and a second pesticidal composition comprising at least one insecticide, wherein the pesticidal activity of the mixture is greater than the sum of the activity of the first pesticidal composition alone and the activity of the second pesticidal composition alone. Methods are provided for screening and identifying synergistic pesticidal mixtures according to the invention.

### RELATED APPLICATION

This application claims the benefit of priority of U.S. Provisional Patent Application No. 61/358,257 filed Jun. 24, 2010, which is expressly incorporated herein by reference in its entirety.

### FIELD OF THE INVENTION

The invention relates to pesticidal mixtures comprising a first pesticidal composition comprising at least two essential oils, a carrier oil, and an emulsifier, and a second pesticidal composition comprising at least one insecticide, wherein pesticidal activity of the mixture is greater than the sum of the activity of the first pesticidal composition alone and the activity of the second pesticidal composition alone.

### INTRODUCTION

The control of pests is important in achieving desired levels of crop efficiency. Pest damage to growing and stored agronomic crops can cause significant reduction in productivity, which can result in increased costs to the consumer. Many products are commercially available and commonly uses for controlling pests, where such products have been used as single agent or mixed agent formulations. However, more efficient pest control compositions and methods are still being sought.

Certain natural products containing pesticidal compositions are described in the literature. For example, pesticidal compositions comprising at least two essential oils, an agriculturally acceptable carrier oil and an emulsifier are known (WO 2007/132224 and EP 1689237 B1). One such product is known as BUGOIL®, which is marketed as a benign insect and mite control agent, and is manufactured by Plant Impact plc (Bamber Bridge, Preston, PR5 BL).

### SUMMARY OF THE INVENTION

The present disclosure provides compositions and methods for synergistic pesticidal mixtures comprising a first pesticidal composition comprising at least two essential oils, a carrier oil, and an emulsifier, and a second pesticidal composition comprising at least one insecticide, wherein the pesticidal activity of the mixture is greater than the sum of the activity of the first pesticidal composition alone and the activity of the second pesticidal composition alone.

The present disclosure provides synergistic pesticidal mixtures comprising a first pesticidal composition and a second pesticidal composition, wherein the pesticidal activity of the mixture exhibits synergistic effects compared with the expected effects of the mixtures based on the sum of the activity of the first pesticidal composition alone and the activity of the second pesticidal composition alone.

The present disclosure provides synergistic pesticidal mixtures comprising a first pesticidal composition comprising at least two essential oils, a carrier oil, and an emulsifier, and a second pesticidal composition comprising at least one insecticide, wherein the first pesticidal composition and the second pesticidal composition are each present in an amount such that the pesticidal activity of the mixture is greater than the sum of the activity of the first pesticidal composition alone and the activity of the second pesticidal composition alone.

The present disclosure provides methods of making and using synergistic pesticidal mixtures comprising a first pesticidal composition comprising at least two essential oils, a carrier oil, and an emulsifier, and a second pesticidal composition comprising at least one insecticide, wherein the pesticidal activity of the mixture is greater than the sum of the activity of the first pesticidal composition alone and the activity of the second pesticidal composition alone.

The present disclosure provides methods of screening and identifying synergistic pesticidal mixtures comprising a first pesticidal composition comprising at least two essential oils, a carrier oil, and an emulsifier, and a second pesticidal composition comprising at least one insecticide, wherein the pesticidal activity of the mixture is greater than the sum of the activity of the first pesticidal composition alone and the activity of the second pesticidal composition alone.

The present disclosure provides pesticidal mixtures comprising a first pesticidal composition comprising at least two essential oils, a carrier oil, and an emulsifier, and a second pesticidal composition comprising at least one insecticide, wherein the pesticidal activity of the mixture allows the use of lower amounts of each component to achieve a desired level of control of the target pest, and/or provides a desired level of control of the target pest sooner after application of the mixture, compared with the amount and/or time required for first pesticidal composition alone, and the amount and/or time required for the second pesticidal composition alone, to achieve the same level of control.

### BRIEF DESCRIPTION OF THE FIGURES

FIG. 1 shows the activity of abamectin (as VERTAN® 1.8 EC) and BUGOIL® against T. urticae, measured as % mortality at 72 hours after application, as disclosed in Example 1; FIG. 1A shows a dose-response curve for abamectin; FIG. 1B shows the effects of BUGOIL® alone and in mixtures with abamectin.

FIG. 2 shows the activity of imidacloprid (as CONFIDOR® 20 LS) and BUGOIL® against T. urticae, measured as % mortality at 72 hours after application, as disclosed in Example 2; FIG. 2A shows a dose-response curve for application of imidacloprid; FIG. 2B shows the effects of BUGOIL® alone and BUGOIL®) in mixtures with imidacloprid.

FIG. 3 shows the activity of indoxacarb (as STEWARD® 30 WG) and BUGOIL® against T. urticae measured as % mortality at 72 hours after application, as disclosed in Example 3; FIG. 3A shows a dose-response curve for application of indoxacarb; FIG. 3B shows the effects of BUGOIL® alone and BUGOIL® in mixtures with indoxacarb.

FIG. 4 shows the activity of lambda-cyhalothrin (KARATE KING® 2.5 WG) and BUGOIL® against T. urticae, measured as % mortality at 72 hours after application, as disclosed in Example 3; FIG. 4A shows a dose-response curve for application of lambda-cyhalothrin; FIG. 4B shows the effects of BUGOIL® alone and BUGOIL® in mixtures with lambda-cyhalothrin.

FIG. 5 shows the activity of spinosad (as SPINTOR® 48 SC) and BUGOIL® against T. urticae, measured as % mortality at 72 hours after application, as disclosed in Example 5; FIG. 5A shows a dose-response curve for application of spinosad; FIG. 5B shows the effects of BUGOIL® alone and BUGOIL® in mixtures with spinosad.

FIG. 6 shows the activity of abamectin (as VERTAN® 1.8 EC) and BUGOIL® against M. persica, measured as % mortality at 72 hours after application, as disclosed in Example 6; FIG. 6A shows a dose-response curve for application of abamectin; FIG. 6B shows the effects of BUGOIL® alone and BUGOIL® in mixtures with abamectin.

FIG. 7 shows the activity of imidacloprid (as CONFIDOR® 20 LS) and BUGOIL® against M. persica, measured as % mortality at 72 hours after application, as disclosed in Example 7; FIG. 7A shows a dose-response curve for application of imidacloprid; FIG. 7B shows the effects of BUGOIL® alone and BUGOIL® in mixtures with imidacloprid.

FIG. 8 shows the activity of indoxacarb (as STEWARD® 30 WG) and BUGOIL® against M. persica, measured as % mortality at 72 hours after application, as disclosed in Example 8; FIG. 8A shows a dose-response curve for application of indoxacarb; FIG. 8B shows the effects of BUGOIL® alone and BUGOIL® in mixtures with indoxacarb.

FIG. 9 shows the activity of lambda-cyhalothrin (as KARATE KING® 2.5 WG) and BUGOIL® against M. persica, measured as % mortality at 72 hours after application, as disclosed in Example 9; FIG. 9A shows a dose-response curve for application of lambda-cyhalothrin; FIG. 9B shows the effects of BUGOIL® alone and BUGOIL® in mixtures with lambda-cyhalothrin.

FIG. 10 shows the activity of spinosad (as SPINTOR® 48 SC) and BUGOIL® against M. persica, measured as % mortality at 72 hours after application, as disclosed in Example 10; FIG. 10A shows a dose-response curve for application of spinosad; FIG. 10B shows the effects of BUGOIL® alone and BUGOIL® in mixtures with spinosad.

FIG. 11 shows the activity of BUGOIL® and lambda cyhalothrin (as KARATE KING® 2.5 WG) against adult whitefly (Trialeurodes sp.) on zucchini plants, at 1, 3, 7, and 14 day after application (1 DAA, 3 DAA, 7 DAA, 14 DAA).

FIG. 12 shows the activity of BUGOIL® and imidacloprid (as CONFIDOR® 20 LS) against adult whitefly (Trialeurodes sp.) on zucchini plants at 1, 3, 7, and 14 day after application (1 DAA, 3 DAA, 7 DAA, 14 DAA).

FIG. 13 shows the activity of BUGOIL® and abamectin (as VERTAN® 1.8 EC (1.8% EC)) against adult whitely Bemisia tabaci on cucumber plants at 1, 3, 7, and 14 day after application (1 DAA, 3 DAA, 7 DAA, 14 DAA).

FIG. 14 shows the activity of BUGOIL® and lambda cyhalothrin (as WARRIOR®) (FIG. 14A), and alone, and BUGOIL® and abamectin (as ZEPHYR® 0.15 EC) (FIG. 14B), against whitefly nymphs on cotton.

FIG. 15 shows the activity of BUGOIL® and imidacloprid (as CLIMAX® 200 SL) against adult whitefly, B. tabaci on eggplant.

FIG. 16 shows the activity of BUGOIL® and imidacloprid (as CLIMAX® 200 SL) against the leafhopper E biggutula on eggplant at 1 hour after application (1HAA), and at 1, 3, 7, and 14 days after application (1 DAA, 3 DAA, 7 DAA, 14 DAA)

### DETAILED DESCRIPTION OF THE INVENTION

The present disclosure provides synergistic pesticidal mixtures comprising a first pesticidal composition comprising at least two essential oils, a carrier oil, and an emulsifier, and a second pesticidal composition comprising at least one insecticide, wherein the pesticidal activity of the mixture is greater than the sum of the activity of the first pesticidal composition alone and the activity of the second pesticidal composition alone.

The present disclosure provides synergistic pesticidal mixtures comprising a first pesticidal composition comprising at least two essential oils, a carrier oil, and an emulsifier, and a second pesticidal composition comprising at least one insecticide wherein the pesticidal activity of the mixture against a target pest is greater than the sum of the activity against the target pest of each pesticidal composition alone.

The present disclosure provides synergistic pesticidal mixtures comprising a first pesticidal composition comprising at least two essential oils, a carrier oil, and an emulsifier, and a second pesticidal composition comprising at least one acaricide wherein the pesticidal activity of the mixture against a mite is greater than the sum of the activity against the mite of each pesticidal composition alone.

The present disclosure provides synergistic pesticidal mixtures comprising a first pesticidal composition comprising at least two essential oils, a carrier oil, and an emulsifier, and a second pesticidal composition comprising at least one insecticide wherein the pesticidal activity of the mixture against an insect is greater than the sum of the activity against the insect of each pesticidal composition alone.

In accordance with one aspect, synergistic pesticidal mixtures as provided herein, which exhibit synergistic effects with respect to the observed pesticidal activity of the mixture when compared with the expected pesticidal effect of the mixture based on the sum of the activity of the first pesticidal composition alone and the activity of the second pesticidal composition alone, allow the use of considerably lower amounts of active ingredients in the synergistic pesticidal mixtures than the amounts that would be required to achieve comparable levels of control using the first pesticidal composition alone, or the second pesticidal composition alone.

In accordance with one aspect, synergistic pesticidal mixtures are provided comprising a first pesticidal composition comprising at least two essential oils, a carrier oil, and an emulsifier, and a second pesticidal composition comprising at least one insecticide, wherein the pesticidal activity of the mixture against a target pest is greater than the sum of the activity of the first pesticidal composition alone and the activity of the second pesticidal composition alone, wherein the synergistic pesticidal mixtures have little or no toxic activity on hosts infested by the target pest. In accordance with a further aspect, synergistic pesticidal mixtures are provided having pesticidal activity against target pests that infest plants, and causing little or no phytotoxicity to host plants of the target pests.

The present disclosure provides pesticidal mixtures comprising a first pesticidal composition comprising at least two essential oils, a carrier oil, and an emulsifier, and a second pesticidal composition comprising at least one insecticide, wherein the pesticidal activity of the mixture allows the use of lower amounts of each component to achieve a desired level of control of the target pest, compared with the amount of first pesticidal composition alone that would be required to achieve the same level of control and/or the amount of second pesticidal composition alone that would be required to achieve the same level of control.

The present disclosure provides pesticidal mixtures comprising a first pesticidal composition comprising at least two essential oils, a carrier oil, and an emulsifier, and a second pesticidal composition comprising at least one insecticide, wherein the pesticidal activity of the mixture provides a desired level of control of the target pest sooner after application of the mixture, compared with the time after application at which the desired amount of control of the target pest is achieved after application of the first pesticidal composition alone, and/or the time after application at which the desired amount of control of the target pest is achieved after application of the second pesticidal composition alone.

The present disclosure provides pesticidal mixtures comprising a first pesticidal composition comprising at least two essential oils, a carrier oil, and an emulsifier, and a second pesticidal composition comprising at least one insecticide, wherein the pesticidal activity of the mixture allows the use of lower amounts of each component to achieve a desired level of control of the target pest and provides a desired level of control of the target pest sooner after application of the mixture, compared with the amount of first pesticidal composition alone and the time after application of the first pesticidal composition alone that would be required to achieve the same level of control of the target pest, and/or the amount of second pesticidal composition alone and the time after application of the second pesticidal composition alone that would be required to achieve the same level of control of the target pest.

### DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

All applications, publications, patents and other references, citations cited herein are incorporated by reference in their entirety. In case of conflict, the specification, including definitions, will control.

As used herein, the singular forms “a”, “and,” and “the” include plural referents unless the context clearly indicates otherwise.

As used herein, all numerical values or numerical ranges include integers within such ranges and fractions of the values or the integers within ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

As used herein, the terms “pest” or “pest” or grammatical equivalents thereof, are understood to refer to organisms, e.g., including pathogens, that negatively affect plants and animals by colonizing, attacking, infesting, or infecting them. It is understood that the terms “pest” or “pest” or grammatical equivalents thereof can refer to organisms that have negative effects by infesting plants and seeds, and commodities such as stored grain.

As used herein, the terms “pesticide” or “pesticidal” or grammatical equivalents thereof, are understood to refer to substances that can be used in the control of agricultural, natural environmental, and domestic/household pests, such as pests referred to as arthropods, insects, arachnids, aphids, leafhoppers, whiteflies, cutworms, borers, fungi, bacteria, and viruses, where the terms not limited to a specific taxonomic classification. The term “pesticide” encompasses, but is not limited to, naturally occurring compounds as well as so-called “synthetic chemical pesticides” having structures or formulations that are not naturally occurring, where pesticides may be obtained by various means including, but not limited to, extraction from biological sources, chemical synthesis of the compound, chemical modification of naturally occurring compounds obtained from biological sources.

As used herein, the terms “insecticidal” and “acaridical” or “aphicidal” or grammatical equivalents thereof, are understood to refer to substances having pesticidal activity against organisms encompassed by the taxonomical classification of root term and also to refer to substances having pesticidal activity against organisms encompassed by colloquial uses of the root term, where those colloquial uses may not strictly follow taxonomical classifications. The term “insecticidal” is understood to refer to substances having pesticidal activity against organisms generally known as insects of the phylum Arthropoda, class Insecta. Further as provided herein, the term is also understood to refer to substances having pesticidal activity against other organisms that are colloquially referred to as “insects” or “bugs” encompassed by the phylum Arthropoda, although the organisms may be classified in a taxonomic class different from the class Insecta. According to this understanding, the term “insecticidal” can be used to refer to substances having activity against arachnids (class Arachnida), in particular mites (subclass Acari/Acarina), in view of the colloquial use of the term “insect.” The term “acaridical” is understood to refer to substances having pesticidal activity against mites (Acari/Acarina) of the phylum Arthropoda, class Arachnida, subclass Acari/Acarina. The term “aphicidal” is understood to refer to substances having pesticidal activity against aphids (Aphididae) of the phylum Arthopoda, class Insecta, family Aphididae. It is understood that all these terms are encompassed by the term “pesticidal” or “pesticide” or grammatical equivalents. It is understood that these terms are not necessarily mutually exclusive, such that substances known as “insecticides” can have pesticidal activity against organisms of any family of the class Insecta, including aphids, and organisms that are encompassed by other colloquial uses of the term “insect” or “bug” including arachnids and mites. It is understood that “insecticides” can also be known as acaricides if they have pesticidal activity against mites, or aphicides if they have pesticidal activity against aphids.

As used herein, the terms “control” or “controlling” or grammatical equivalents thereof, are understood to encompass any pesticidal (killing) activities or pestistatic (inhibiting, repelling, deterring, and generally interfering with pest functions to prevent the damage to the host plant) activities of a pesticidal composition against a given pest. Thus, the terms “control” or “controlling” or grammatical equivalents thereof, not only include killing, but also include such activities as repelling, deterring, inhibiting or killing egg development or hatching, inhibiting maturation or development, and chemisterilization of larvae or adults. Repellant or deterrent activities may be the result of compounds that are poisonous, mildly toxic, or non-poisonous to pests, or may act as pheromones in the environment.

First Pesticidal Composition

In a non-limiting exemplary embodiment, synergistic pesticidal mixtures are provided comprising a first pesticidal composition comprising at least two essential oils, a carrier oil, and an emulsifier, and a second pesticidal composition comprising at least one insecticide, wherein the pesticidal activity of the mixture against a pest is greater than the sum of the activity against the pest of each pesticidal composition alone.

In a non-limiting exemplary embodiment of the pesticidal mixtures provided herein, a first pesticidal composition of the present invention may be a pesticidal composition comprising at least two essential oils, an agriculturally acceptable carrier oil and an emulsifier as described, e.g., in EP 1689237 B1 or WO 2007/132224, the entire contents of which are hereby incorporated by reference. Essential oils suitable for use in the present invention include, but are not limited to, tagetes (or, tagettes) oil obtainable from various Tagetes (marigold) species including T. erecta or T. minuta, and thymol-containing oils including, but not limited to, thyme oil obtainable from various Thymus (thyme) species including T. vulgaris, basil oil obtainable from various Ocimum (sweet basil) species including Ocimum basilicium, anabasis oil obtainable from various Anabasis species, carroway oil obtainable from various Carum species, lavender oil obtainable from various Lavendula species, marjoram (organum) oil obtainable from various Organum species, and palmerosa oil obtainable from various Cymbopogon species such as Cymbopogon martini. It is understood that thyme oil is a particularly suitable thymol-containing essential oil., but others include Anabasis, carum, lavendula, Ocimum, and organum oils. It is further understood that one or more isolated components of these oils may be utilised, provided these have properties that contribute to the pesticidal propertieso of the first pesticidal composition. For example, thyme oil from Thymus vulgaris comprises a mixture of thymol, caracrol, cymol, linalool, terpin-4-ol an monoterpenoids, where any of these components or mixtures thereof may be used in the first pesticidal composition. Components of tagetes oil, e.g., from Tagetes erecta or Tagetes minuta, include dihydrotagetone, thiophenes and ocimene, or which dihydrotagetone is the most important component.

Agriculturally acceptable carrier oils suitable for use in the present invention include, but are not limited to, a vegetable oil such as including canola oil (also known as Oil Seed Rape oil, OSR), maize (corn) oil, sunflower oil, cottonseed oil, and soybean oil. Emulsifiers are present in an amount which is sufficient to ensure that the composition has the desired miscibility with water, where emulsifiers suitable for use in the present invention include, but are not limited to, any known agriculturally acceptable emulsifier such as a surfactant, typically alkylaryl sulphonates, ethoxylated alcohols, polyalkoxylated butyl ethers, calcium alkyl benzene sulphonates, polyalkylene glycol ethers, butyl polyalkylene oxide block copolymers, or nonyl phenol emulsifiers such as TRITON N57™, polyoxyethylene sorbitan esters such as polyoxyethylene sorbitan monolaurate (sold by ICI under the trade name “TWEEN™”), or natural organic emulsifiers such as coconut oil or coconut oil products (e.g., coconut diethanolamide), castor oil or castor oil products (e.g., Marlowet®), or palm oil products such as lauryl stearate. In non-limiting embodiments, the emulsifier may be present in amounts of from 1 to 20% w/w, suitably up to 10% w/w and in particular about 5% or 6% w/w.

In a further non-limiting exemplary embodiment of the pesticidal mixtures provided herein, a first pesticidal composition of the present invention may be a pesticidal composition as described, in EP 1689237 B1, comprising (i) a mixture of tagetes oil and thyme oil in a ratio of from 3:1 to 1:3, wherein the total amount of such oil or mixture present does not exceed 10% w/w; (ii) an agriculturally acceptable carrier oil and (iii) an emulsifier. Further non-limiting embodiments are provided wherein the pesticidal composition comprises no more than 5% w/w of component (i), where certain such compositions contain no more than 1.5% w/w of component (i). Further non-limiting embodiments are provided wherein the carrier oil (component (ii) of the pesticidal composition) may be canola oil, sunflower oil, cottonseed oil, palm oil, or soybean oil. Further non-limiting embodiments are provided wherein the pestidical composition may comprise additional components including, but not limited to, compounds which remediate symptoms of viral infection such as salicylates (e.g., salicyclic acid, salicylic acid esters, methyl salicylate), or compounds with antiviral activity such as jasmonates (e.g., jasmonic acid and jasmonic acid derivatives, C₁₋₁₀ alklyl esters of jasmonic acid, methyl jasmonate). In one non-limiting embodiment, salicylates may be contained in an essential oil which includes salicylic acid or salicylates such as wintergreen oil, as well as oils from Chenopodium, Erythroxylum, Eugenia, Gaultheria, Myristica, Syzygium, Xanthophyllum, Cinnamonium, Gualtheria, Gossypium and Mentha species. Without wishing to be limited by this theory, it is understood that additional components such as salicylates may act as synergizers of the effects of active ingredients.

In a further non-limiting exemplary embodiment of the pesticidal mixtures provided herein, the first pesticidal composition of the present invention may be a pesticidal composition marketed under the names BUGOIL®, and/or Suprila®, and/or Marigold® (Plant Impact plc (Bamber Bridge, Preston, PR5 BL), comprising substances that function as carriers, emulsifer, active substances, and optionally, synergizers. An exemplary BUGOIL® formulation contains a mixture of canola oil as a carrier oil, Tween20® as an emulsifer, tagettes (or, tagetes) oil as an active substance, thyme oil as an active substance, and wintergreen oil as a synergizer. A formulation used in the non-limiting Examples below contains canola oil (93.799% w/w) as a carrier oil, Tween20® (5.000% w/w) as an emulsifer, tagettes oil (0.600% w.w) as an active substance, thyme oil (0.600% w/w) as an active substance, and wintergreen oil (0.0001% w/w) as a synergizer.

Second Pesticidal Composition

The present disclosure provides pesticidal mixtures comprising a first pesticidal composition comprising at least two essential oils, a carrier oil, and an emulsifier, and a second pesticidal composition comprising at least one insecticide, wherein second pesticidal composition contains an insecticidally active compound that can selected by one of skill in the art. Non-limiting exemplary embodiments include one or more pesticidal compositions having activity against sucking pests including, but not limited to, mites, aphids, whitefly, thrips, scales (scale insects), leafhoppers, and other targets that may be identified by one of skill in the art.

Suitable insecticidal compounds include, but are not limited to:

Abamectin, a mixture of avermectins, e.g., B₁ₐ (80%) and B_{1b} (20%): as follows:
avermectin B₁ₐ (80%), (2aE,4E,8E)-(5′S,6S,6′R,7S,11R,13S,15S,17aR,20R,20aR,20bS)-6′-[(S)-sec-butyl]-5′,6,6′,7,10,11,14,15,17a,20,20a,20b-dodecahydro-20,20b-dihydroxy-5′,6,8,19-tetramethyl-17-oxospiro[11,15-methano-2H,13H,17H-furo[4,3,2-pq][2,6]benzodioxacyclooctadecin-13,2′-[2H]pyran]-7-yl2,6-dideoxy-4-O-(2,6-dideoxy-3-O-methyl-α-L-arabino-hexopyranosyl)-3-O-methyl-α-L-arabino-hexopyranoside; and
avermectin B_{1b} (20%), (2aE,4E,8E)-(5′S,6S,6′R,7S,11R,13S,15S,17aR,20R,20aR,20bS)-5′,6,6′,7,10,11,14,15,17a,20,20a,20b-dodecahydro-20,20b-dihydroxy-6′-isopropyl-5′,6,8,19-tetramethyl-17-oxospiro[11,15-methano-2H,13H,17H-furo[4,3,2-pq][2,6]benzodioxacyclooctadecin-13,2′-[2H]pyran]-7-yl2,6-dideoxy-4-O-(2,6-dideoxy-3-O-methyl-α-L-arabino-hexopyranosyl)-3-O-methyl-α-L-arabino-hexopyranoside;
Imidacloprid, (2E)-1-[(6-chloro-3-pyridinyl)methyl]-N-nitro-2-imidazolidinimine;
Indoxacarb, methyl (4aS)-7-chloro-2,5-dihydro-2-[[(methoxycarbonyl)[4-(trifluoromethoxy)phenyl]amino]carbonyl] indeno[1,2-e][1,3,4]oxadiazine-4a(3H)-carboxylate;
Lambda-cyhalothrin, equal quantities of isomeric mixture of (R)-α-cyano-3-phenoxybenzyl (1S)-cis-3-[(Z)-2-chloro-3,3,3-trifluoropropenyl]-2,2-dimethylcyclopropanecarboxylate; and (S)-α-cyano-3-phenoxybenzyl (1R)-cis-3-[(Z)-2-chloro-3,3,3-trifluoropropenyl]-2,2-dimethylcyclopropanecarboxylate;
Spinosad, a mixture of 50%-95% (2R,3aS,5aR,5bS,9S,13S,14R,16aS,16bR)-2-[(6-deoxy-2,3,4-tri-O-methyl-α-L-mannopyranosyl)oxy]-13-[[(2R,5S,6R)-5-(dimethylamino)tetrahydro-6-methyl-2H-pyran-2-yl]oxy]-9-ethyl-2,3,3a,5a,5b,6,9,10,11,12,13,14,16a,16b-tetradecahydro-14-methyl-1H-as-indaceno[3,2-d]oxacyclododecin-7,15-dione; and 5%-50%
(2S,3 aR,5aS,5bS,9S,13S,14R,16aS,16bS)-2-[(6-deoxy-2,3,4-tri-O-methyl-α-L-mannopyranosyl)oxy]-13-[[(2R,5S,6R)-5-(dimethylamino)tetrahydro-6-methyl-2H-pyran-2-yl]oxy]-9-ethyl-2,3,3a,5a,5b,6,9,10,11,12,13,14,16a,16b-tetradecahydro-4,14-dimethyl-1H-as-indaceno[3,2-d]oxacyclododecin-7,15-dione,
   and other compounds having insecticidal activity that can be readily identified by one of skill in the art.

Suitable insecticidal compounds are available as commercial products from many suppliers. Abamectin is commercially available in products sold under trade names including, but not limited to, Abba®, Affirm®, AGRI-MEK®, Avid®, Dynamec®, MK 936®, VERTAN®, Vertimec®, ZEPHYR®, and Cure®. Imidacloprid is commercially available in products sold under trade names including, but not limited to, Admire®, Advantage®, Condifor®, Kohinor®, Gaucho®, Hachikusan®, Marathon®, Merit®, Premier®, Premise®, PROVADO®, Prothor®, and Winner®. Indoxacarb is commercially available in products sold under trade names including, but not limited to, Advion®, Arilon®, Avaunt®, and STEWARD®. Lambda-cyhalothrin is commercially available in products sold under trade names including, but not limited to, Charge®, Excaliber®, Grenade®, Hallmark®, Icon®, Karate®, KARATE KING®, Matador®, OMS 0321®, PP321®, Saber®, Samurai®, and Sentinel®. Spinosad is commercially available in products sold under trade names including, but not limited to, Conserve®, Entrust®, Intrepid®, SPINTOR®, Success®, and Tracer®. One skilled in the art can identify and select one or more commercial products containing insecticdal compounds for use in the present invention based on evaluation of factors including, but not limited to, pest(s) to be controlled, crop, growing conditions, application methods, and other factors.

Pesticidal Mixtures

Pesticidal mixtures as provided herein may further comprise an inert carrier. Suitable inert carrier can be solid carriers, liquid carriers and the like which are utilized for usual pesticidal formulations such as emulsifiable concentrates, wettable powders, or flowables. Pesticidal mixtures as provided herein may optionally comprise formulation auxiliaries such as oils, surfactants, dispersants, adhesive agents, stabilizers and propellants (formulated to oil solutions). Pesticidal mixtures as provided herein may be formulated as aqueous suspensions, aqueous emulsions, microcapsule formulations, emulsifiable concentrates, wettable powders, or flowables, granules, dusts, aerosols, ULV formulations, or other formulations that can be selected and prepared by one of skill in the art to yield formulations having suitable properties for use in accordance with the present invention.

Pesticidal mixtures as provided here, and methods for making and using pesticidal mixtures as provided herein, can be applied for controlling various pests such as agricultural and forestry pests, ectoparasites of animals and hygienically unfavorable pests. Pesticidal mixtures as provided here can be applied for controlling arthropod pests (phylum Arthopoda) such as arachnids (class Arachnida), in particular mites (subclass Acari/Acarina), and insect pests (class Insecta), including but not limited to lepidoptera (order Lepidoptera), aphids (family Aphididae), and hemiptera (order Hemiptera).

Pesticidal mixtures as provided herein can be applied for controlling various types of mites, in particular spider mites (family Tetranychidae), including but not limited to, the common spider mite/two-spotted spider mite (Tetranychus urticae), Kanzawa spider mite (Tetranychus kanzawai) the fruit tree spider mite (Panonychus ulmi), the citrus spider mite (Panonychus citri), the citrus rust mite (Phylocoptruta oleivora), and Oligonychus spp. Pesticidal mixtures as provided herein can be applied for controlling various mites of families including, but not limited to, family Eriophyidae, e.g. Aculops pelekassi (pink citrus rust mite) and Calacarus carinatus (purple tea mite), family Tarsonemidae, e.g. Polyphagotarsonemus latus, false spider mites of family Tenuipalpidae, e.g. Brevipalpus chilensi, family Tuckerellidae, family Ixodidae, e.g. Haemaphysalis flava (Japanese tick), Haemaphysalis flava (yellow tick), Ixodes ovatus and Ixodes persulcatus, family Acaridae, e.g. Tyrophagus putrescentiae (copra mite), family Dermanyssidae, e.g. Dermatophagoides farinae (American house dust mite), Dermatophagoides ptrenyssnus, family Cheyletidae, e.g. Cheyletus eruditus, Cheyletus fortis, Cheyletus malaccensi and Cheyletus moorei, and other mites, especially spider mites, that may be identified by one of skill in the art for use in accordance with the present invention.

Pesticidal mixtures as provided herein can be applied for controlling various types of aphids (family Aphididae), e.g., Myzus persicae (green peach aphid), Aphis gossypii (cotton aphids), Aphis citricola, Lipaphis pserudobrassicae (turnip aphid), Nippolachnus piri (pear green aphid), Toxoptera aurantii (black citrus apid) and Toxoptera ciidius (brown citrus aphid), and other aphids that may be identified by one of skill in the art for use in accordance with the present invention.

Pesticidal mixtures as provided herein can be applied for controlling hemipteran pests such as pests of the family Delphacidae (planthoppers), e.g. Laodelphax striatellus (small brown planthopper), Nilaparvata lugens (brown planthopper) and Sogatella furcifera (white-backed rice planthopper), pests of the family Cicadellidae (leafhoppers), e.g., pests of the genus Empoasca (Empoasca sp.) including but not limited to Empoasca biggutula, pests of the family/subfamily Deltocephalidae (leafhoppers), e.g., pests of the genus Nephotettix (Nephotettix sp.) including but not limited to Nephotettix cincticeps and Nephotettix virescens, pests of the family Cicadellidae (leafhoppers) pests of the family Pentatomidae (stink bugs), e.g. Nezara antennata (green stink bug), Cletus punctiger, Riptortus clavetus (bean bug) and Plautia stali (oriental stink bug), pests of the family Aleyrodidae (whiteflies), e.g. pests of the genus Trialeurodes (Trialeurodes sp.) including but not limited to Trialeurodes vaporariorum (greenhouse whitefly), pests of the genus Bemisia (Bemisia sp.) including but not limited to Bemisia tabaci (sweetpotato whitefly) and Bemisia argentifolli (silverleaf whitefly), pests of the family Diaspididae (scales), e.g. Aonidiella aurantii (Calif. red scale), Comstockaspis perniciosa (San Jose scale), Unaspis citri (citrus snow scale), Pseudaulacaspis pentagona (white peach scale), Saissetia oleae (brown olive scale), Lepidosaphes beckii (purple scale), Ceroplastes rubens (red wax scale) and Icerya purchasi (cottonycushion scale), pests of the family Tingidae (lace bugs), pests of the family Psyllidae (suckers), and other hemipteran pests that may be identified by one of skill in the art for use in accordance with the present invention.

The pesticidal mixture may also be used in the control larvae and eggs.

The present disclosure provides methods of pest control, including contacting a target pest with an amount of pesticidal mixture composition as described herein, sufficient to result in control of the pest. Non-limiting exemplary methods provided herein include, applying the pesticidal mixture composition to a target pest, applying the pesticidal mixture to a substrate associated with a target pest, applying the pesticidal mixture to a site of target pest infestation, wherein the pesticidal mixture is applied in an amount sufficient to control the pest. Non-limiting exemplary methods provided herein include applying the pesticidal mixture to a site of target pest infestation in an amount that is sufficient to control the pest and does not damage the infested host tissue(s). Non-limiting exemplary methods provided herein include applying the pesticidal mixture to a site of potential target pest infestation, e.g., by applying to a noninfested host, in an amount that is sufficient to prevent or reduce the pest infestation and does not damage the host tissue(s).

The pesticidal mixtures provided herein may be used for the protection of crops against agricultural pests. Pesticidal mixtures as provided herein are suitable for use on most crops, including field crops, orchard produce, and greenhouse-grown crops. Crops suitable for use with pesticide mixtures as provided herein include, but are not limited to, cereals such as wheat, barley, rye, oats, rice, maize and sorghum, tree fruits such as pome fruits, stone fruit, soft fruit, including apples, pears, plums, peaches, almonds, and cherries, berries such as strawberries, raspberries and blackberries, leguminous crops such as beans, lentils, peas and soybeans, oil plants such as rape, mustard, poppy, olives, sunflowers, coconut, castor oil, cocoa and groundnuts, beets such as sugar beet and fodder beet, cucurbitaceae such as marrows, cucumbers zucchinis, and melons, fiber plants such as cotton, flax, hemp and jute, citrus fruits, such as oranges, lemons, grapefruit and mandarins, vegetables, such as spinach, lettuce, asparagus, cabbages, carrots, onions, peppers, tomatoes, potatoes, eggplants, lauraceae such as avocado, and other crops such as cinnamon, camphor, tobacco, nuts, coffee, sugar cane, tea, vines, hops, bananas, natural rubber plants and ornamentals; as well as seeds of such crops.

Pesticidal Mixtures

The present disclosure provides synergistic pesticidal mixtures comprising a first pesticidal composition comprising at least two essential oils, a carrier oil, and an emulsifier, and a second pesticidal composition comprising at least one insecticide having acaricidal activity (acaricide) wherein the pesticidal activity of the mixture against the target mite is greater than the sum of the activity against the mite of each pesticidal composition alone. In a non-limiting exemplary embodiment, synergistic pesticidal mixtures are provided comprising a first pesticidal composition comprising at least two essential oils, a carrier oil, and an emulsifier, and a second pesticidal composition comprising at least one acaricide wherein the pesticidal activity of the mixture against the two spotted red spider mite, Tetranychus urticae, is greater than the sum of the activity against T. urticae of each pesticidal composition alone.

The present disclosure provides synergistic pesticidal mixtures comprising a first pesticidal composition comprising at least two essential oils, a carrier oil, and an emulsifier, and a second pesticidal composition comprising at least one insecticide having aphicidal activity, wherein the pesticidal activity of the mixture against the target aphid, is greater than the sum of the activity against the target aphid of each pesticidal composition alone. In a non-limiting exemplary embodiment, synergistic pesticidal mixtures are provided comprising a first pesticidal composition comprising at least two essential oils, a carrier oil, and an emulsifier, and a second pesticidal composition comprising at least one aphicide, wherein the pesticidal activity of the mixture against the green peach aphid, Myzus persicae is greater than the sum of the activity against M. persicae of each pesticidal composition alone.

In non-limiting exemplary embodiments, studies were carried out to evaluate the insecticidal potency of pesticidal mixtures comprising BUGOIL® mixed with selected pesticides against the two spotted spider mite Tetranychus urticae and the green peach aphid Myzus persicae under laboratory conditions. In the non-limiting exemplary embodiments described in the Examples below, the expected effects of the selected pesticides when used alone against the red spider mite or the green peach aphid, were based on their commercial usage, and none of the selected pesticides showed unexpected effects when used alone.

In the non-limiting exemplary embodiments described in the Examples below, pesticides that were known to be effective acaricides were demonstrated to be active against T. urticae alone, and in mixtures with BUGOIL®. In the non-limiting exemplary embodiments described in the Examples below, abamectin and spinosad, in product formulations that are commercially available, showed expected activity against T. urticae when used alone, and showed greatly enhanced effects when mixed with BUGOIL® in tests against the spider mite T. urticae, under laboratory conditions.

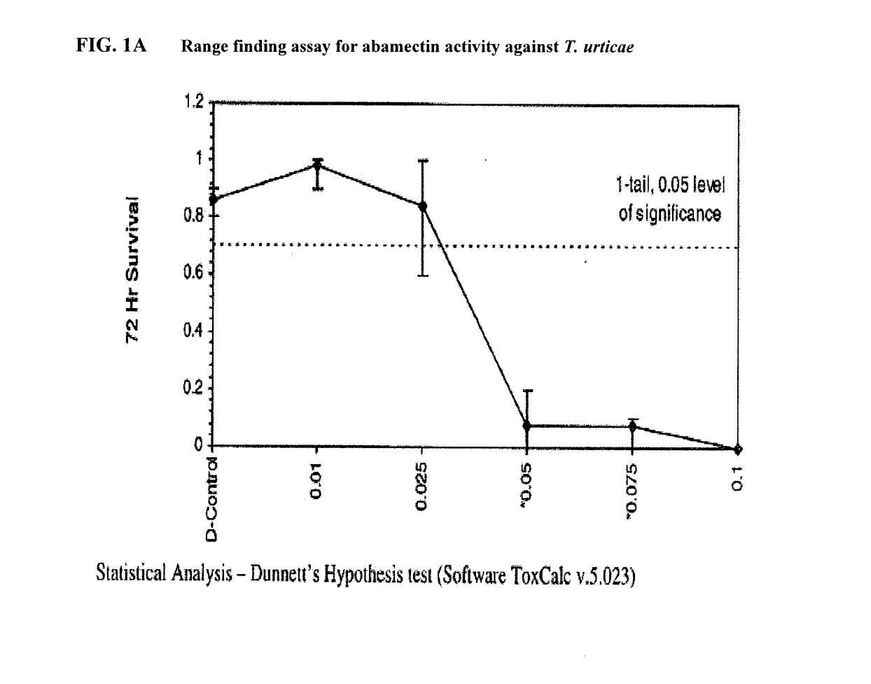

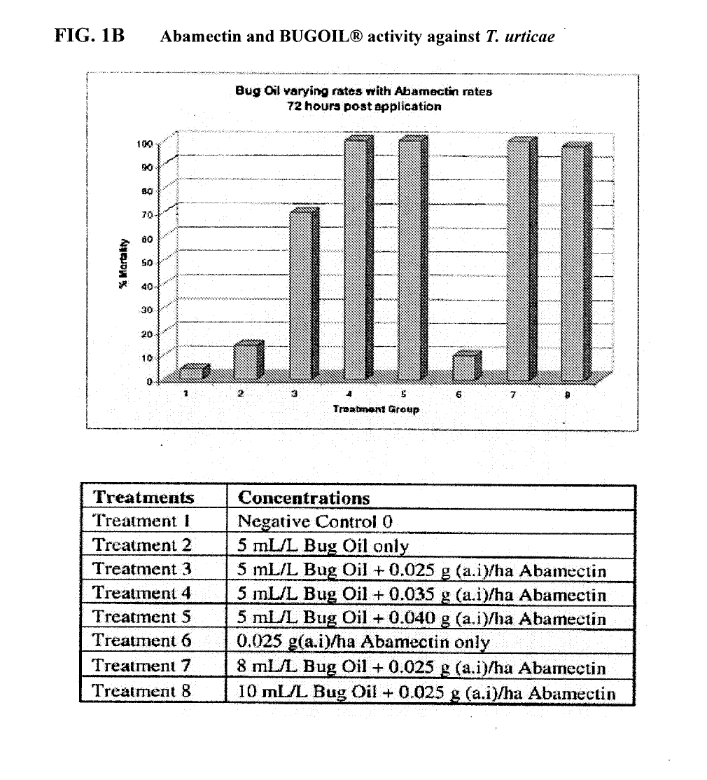

In the non-limiting exemplary embodiments described in the Examples below, mixtures of abamectin and BUGOIL® consistently showed synergistic acaricidal effects, where abamectin is well-known as an acaricide that also significantly reduces female fecundity and can be effective against offspring when applied to the eggs. As demonstrated in Example 1, illustrated in FIG. 1, and summarized in Tables I and II, abamectin showed acaricidal activity against T. urticae when used alone. As demonstrated in Example 1, illustrated in FIG. 1, an summarized in Tables II and III mixtures of abamectin with BUGOIL® increased mortality rates to levels that were significantly greater than the predicted (theoretical) sum of the known activity of the abamectin in the mixture and the known activity of the BUGOIL® in the mixture, indicating synergistic effects of mixtures of abamectin with BUGOIL®.

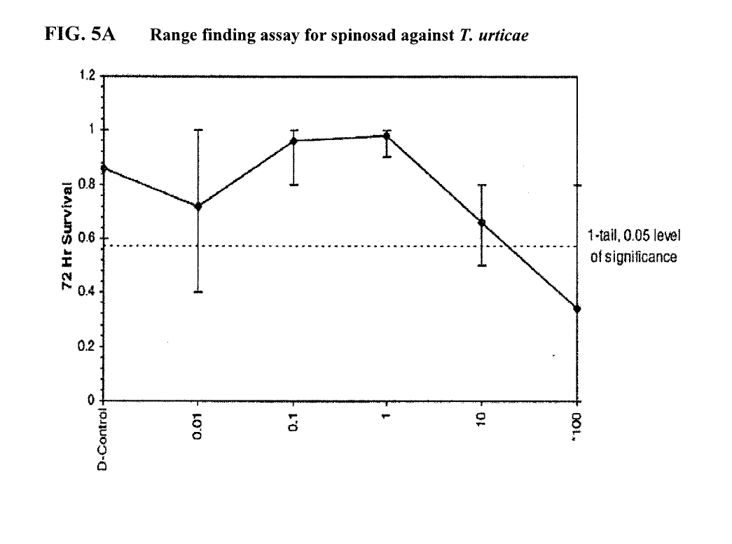

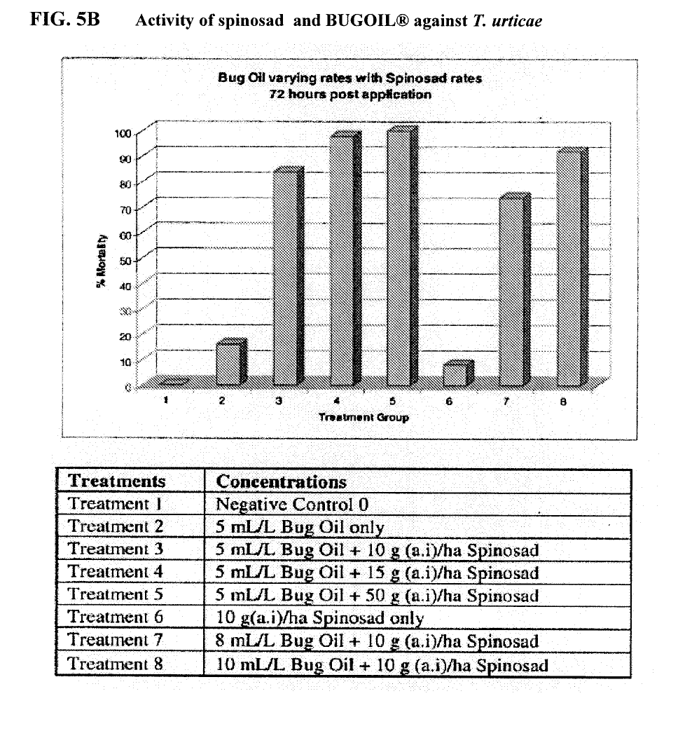

In the non-limiting exemplary embodiments described in the Examples below, mixtures of spinosad and BUGOIL® consistently showed synergistic acaricidal effects, where spinosad has been shown to work well alongside biological control of the red spider mite as it has low toxicity to beneficial arthropods, and may have some systemic properties and is reported to have effects on female fecundity. As demonstrated in Example 5, illustrated in FIG. 5, and summarized in Tables I and II, spinosad showed acaricidal activity against T. urticae when used alone. As demonstrated in Example 5, illustrated in FIG. 5, and summarized in Tables II and III, mixtures of spinosad with BUGOIL® increased mortality rates to levels that were significantly greater than the predicted (theoretical) sum of the known activity of the spinosad in the mixture and the known activity of the BUGOIL® in the mixture, indicating synergistic effects of mixtures of spinosad with BUGOIL®.

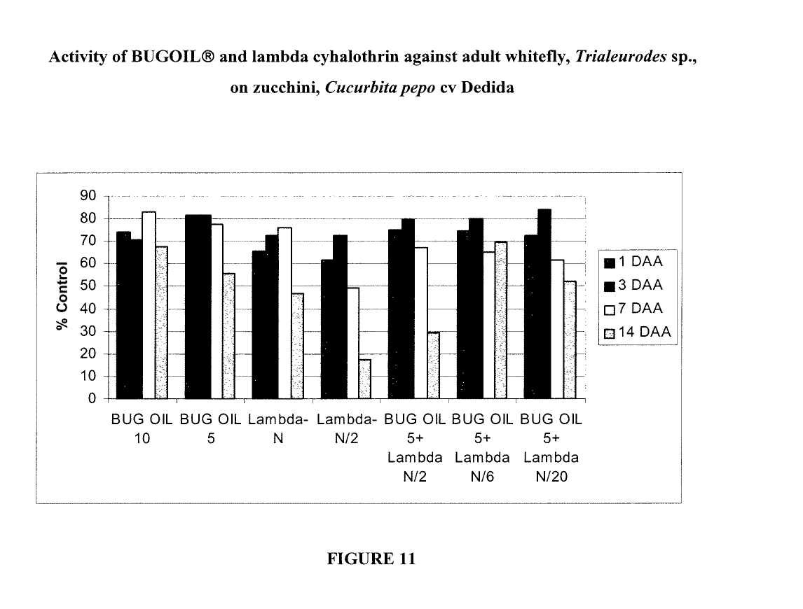

In the non-limiting exemplary embodiments described in the Examples below, mixtures of BUGOIL® with lambda cyhalothrin, or with imidacloprid, or with abamectin, provided slight improvements in activity against adult whiteflies, compared with the activity of each product alone (Example 14 and FIGS. 11, 12, 13). Mixtures of BUGOIL® with imidacloprid consistently gave better control than either product alone.

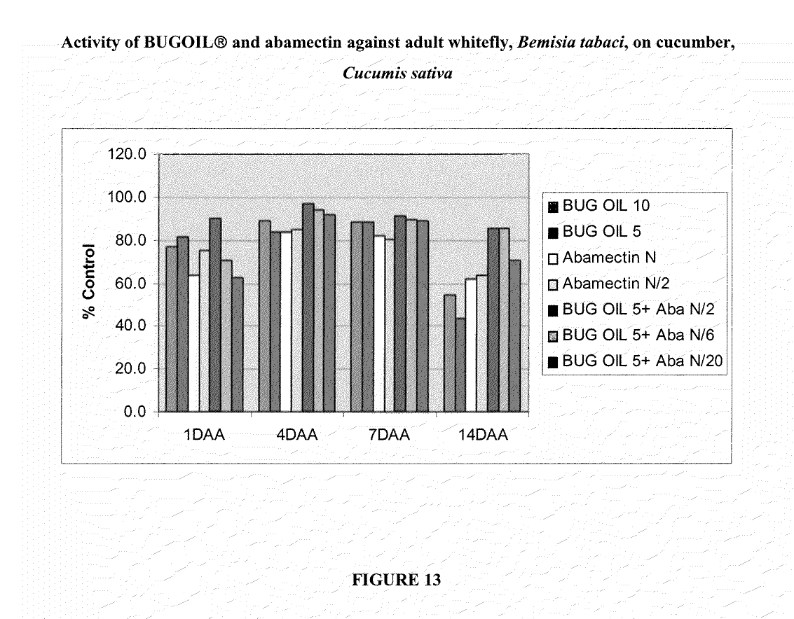

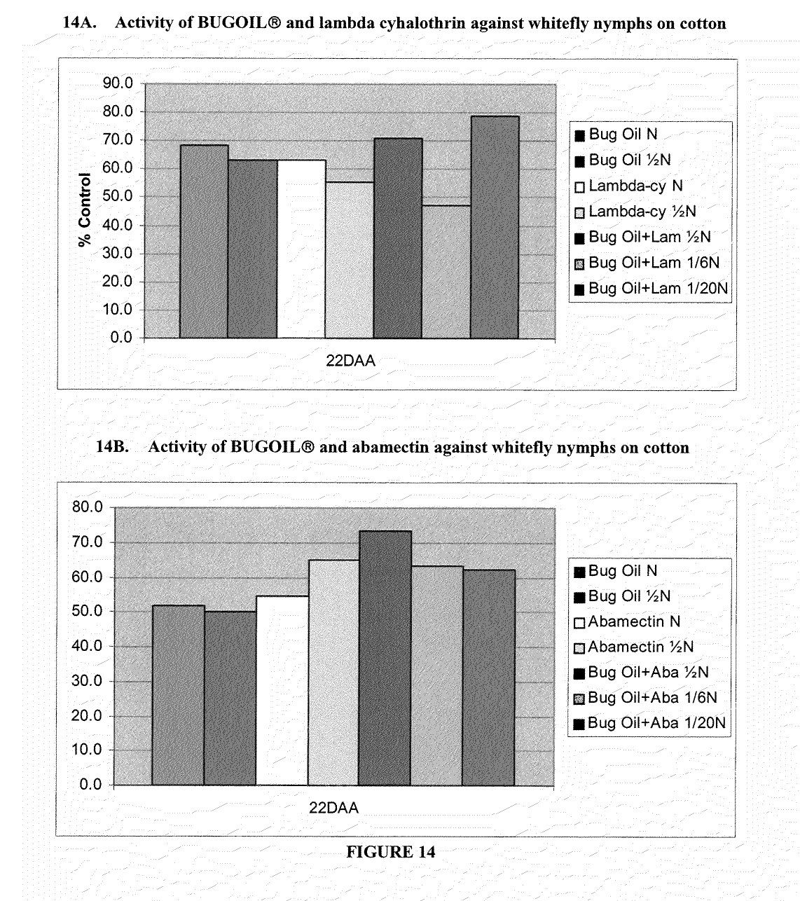

In the non-limiting exemplary embodiments described in the Examples below, mixtures of BUGOIL® with abamectin improved the persistence of the effect on control of whiteflies on different crops (Examples 14, 15, 16, and FIGS. 13, 15, and 16).

In the non-limiting exemplary embodiments described in the Examples below, reduced rates of BUGOIL® with abamectin applied in mixtures achieved improved control of sucking insect pests and lepdopteran pests, and improved persistance of effect (Example 16).

Synergistic Effects

Synergism has been described as “the cooperative action of two components of a mixture, such that the total effect is greater or more prolonged than the sum of the effects of the two (or more) taken independently” (see P. M. L. Yames, Neth. J Plant Pathology 1964, 70, 73-80). Generally speaking, a synergistic effect is understood to exist whenever the action of a mixture or combination of components is greater than the sum of the action of each of the components alone. Therefore, a synergistic mixture of components has an action that is greater than the sum of the action of each component alone. The present disclosure provides pesticidal mixtures of components wherein the pesticidal mixture has pesticidal activity that exceeds the calculated or predicted value of the activity of the mixture based on the known activity of each component, such that pesticidal mixtures provided herein have a synergistic pesticidal effect.

It is understood that a synergistic effect, wherein measured activity of a mixture that exceeds the calculated or predicted value of the activity of the mixture based on the known activity of each component, is different from a pesticide adjuvant effect. Pesticide adjuvants are understood to be chemicals that are added to pesticide formulations in order to enhance the effectiveness of the active ingredient, where adjuvants are not intended to be pesticidal. Pesticide adjuvants have been defined as “[a]ny substance, other than water, without significant pesiticidal properties, which enhances or is intended to enchance, the effectiveness of a pesticide when itis added to the pesticide” (Thacker, 2000, Pesticide Adjuvants, Biol. Sci. AGR 183 (pages 1-7) citing regulatory definitions of the United Kingdom). Ajuvants include, but are not limited to, acidifier, activators, surfactants, anti-foam agents, anti-evaporants, buffers, penetrating agents, compatability agents, defoaming agents, deposition agents, drift control agents, emulsifiers, extenders, foaming agents, humectants, mineral oils, vegetable oils, speaders, stickers, wetters, and water conditioners.

Well-known methods for determining whether synergy exists include the Colby method, the Tammes method and the Wadley method, all of which are described below. Any one of these methods may be used to determine if synergy exists between the compounds A and B. In the Colby method, also referred to as the Limpels method, the action to be expected (E) for a given active ingredient combination obeys the so-called Colby formula. According to Colby, the expected (additive) action of active ingredients A and B at application rates of m and n g/ha or in a concentration of m and n ppm is:

E=(X+Y)−(X*Y/100)

where X is the kill rate, expressed as a percentage of the untreated control, when employing active compound A at an application rate of m g/ha or in a concentration of m ppm, Y is the kill rate, expressed as a percentage of the untreated control, when employing active compound B at an application rate of n g/ha or in a concentration of n ppm; where ppm equals the milligrams of active ingredient (=a.i.) per liter of spray mixture. If the ratio R defined as the action actually observed (O) divided by the expected action (E) is >1 then the action of the combination is superadditive, i.e. there is a synergistic effect. For a more detailed description of the Colby formula, see Colby, S. R. “Calculating synergistic and antagonistic responses of herbicide combination,” Weeds, Vol. 15, pages 20-22; 1967; see also Limpel et al., Proc. NEWCC 16: 48-53 (1962).

The Tammes method uses a graphic representation to determine whether a synergistic effect exists. See “Isoboles, a graphic representation of synergism in pesticides,” Netherlands Journal of Plant Pathology, 70 (1964) p. 73-80.

The Wadley method is based on comparison of an observed ED₅₀ value (i.e. dose of a given compound or combination of compounds providing 50% pest control) obtained from experimental data using the dose response curves and an expected ED₅₀ calculated theoretically from the formula:

$${{ED}_{50} ( A + B )}_{expected} = \frac{a + b}{\frac{a}{{{ED}_{50} ( A )}_{observed}} + \frac{b}{{{ED}_{50} ( B )}_{observed}}}$$

wherein a and b are the weight ratios of compound A and B in the mixture and ED_{50 observed} is the experimentally determined ED₅₀ value obtained using the dose response curves for the individual compounds. The ratio ED₅₀(A+B)_{expected}/ED50(A+B)_{observed} expresses the factor of interaction (F) (synergy factor). In case of synergism, F is >1. The same formula applies when LD₅₀ values are used, i.e. lethal dose, as well as EC₅₀ values, i.e. effective concentration, and LC₅₀ values, i.e. lethal concentration. For a more detailed description of the Wadley method, see Levi et al., EPPO-Bulletin 16, 1986, 651-657.

An alternative approach as mentioned by D. L. Richer (Pesticide Science, 1987, 19, 309-315, especially p. 313) to determine synergy is based on purely observed values rather than observed and theoretical calculated values as used in the previously mentioned methods. In this alternative method the effect of a given rate of the mixture A and B is compared with the effect of the same rate of each of A and B used alone. If synergism exists, the observed effect of the mixture will be greater than the observed effect of either component used alone:

E_{observed}(xA+yB)>E_{observed}(x+y)A, and

E_{observed}(xA+yB)>E_{observed}(x+y)B

wherein x and y are the quantities of A and B in the mixture. If the actual kill rate exceeds the calculated value, the action of the combination is understood to be superadditive, i.e. a synergistic effect is present.

Use of Mixtures to Reduce Application Rates of Each Component and/or Improve Timing of Control

The present disclosure provides pesticidal mixtures comprising a first pesticidal composition comprising at least two essential oils, a carrier oil, and an emulsifier, and a second pesticidal composition comprising at least one insecticide, wherein the pesticidal activity of the mixture allows the use of lower amounts of each component to achieve a desired level of control of the target pest, compared with the amount of first pesticidal composition alone that would be required to achieve the same level of control and/or the amount of second pesticidal composition alone that would be required to achieve the same level of control.

The present disclosure provides pesticidal mixtures comprising a first pesticidal composition comprising at least two essential oils, a carrier oil, and an emulsifier, and a second pesticidal composition comprising at least one insecticide, wherein the pesticidal activity of the mixture provides a desired level of control of the target pest sooner after application of the mixture, compared with the time after application at which the desired amount of control of the target pest is achieved after application of the first pesticidal composition alone, and/or the time after application at which the desired amount of control of the target pest is achieved after application of the second pesticidal composition alone.

The present disclosure provides pesticidal mixtures comprising a first pesticidal composition comprising at least two essential oils, a carrier oil, and an emulsifier, and a second pesticidal composition comprising at least one insecticide, wherein the pesticidal activity of the mixture allows the use of lower amounts of each component to achieve a desired level of control of the target pest and provides a desired level of control of the target pest sooner after application of the mixture, compared with the amount of first pesticidal composition alone and the time after application of the first pesticidal composition alone that would be required to achieve the same level of control of the target pest, and/or the amount of second pesticidal composition alone and the time after application of the second pesticidal composition alone that would be required to achieve the same level of control of the target pest.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include aspects that are not expressly included in the invention are nevertheless disclosed herein.

Various non-limiting exemplary embodiments of the invention have been described, where it is understood that embodiments may be made without departing from the spirit and scope of the invention. Accordingly, the following examples are intended to illustrate but not limit the scope of invention described in the claims.

### EXAMPLES

Studies as described below were carried out to assess the efficacy of BUGOIL® when mixed with selected commercial formulations of insecticides.

Various insecticidal formulations were selected for testing, knowing that in some cases there would be no activity against certain test organisms, thereby providing additional control experiments and/or providing an opportunity to observe improved activity in combinations.

For the laboratory experiments described in Examples 1-13, host plants were infested with target organisms as follows: Phaseolus vulgaris, (French bean) were infested with Tetranychus urticae (two spotted red spider mite, or red spider mite) adults under laboratory conditions and Brassica rapa chinensis, (Chinese Cabbage) were infested with Myzus persicae (green peach aphid) adults. Test arenas consisted of excised leaf discs of the host plant infested with the target organism. A track sprayer was used to spray the test solutions at an application volume of 200 L/ha, where each test solution had a concentration of BUGOIL® other active ingredient as set forth in the present examples and figures. Assessment of mortality, any other sub lethal effects, and phytotoxicity were carried out at 24 hours (1 day after application (DAA)) and 72 hours post application (3DAA) for the experiments described in Examples 1-13.

For the field experiments described in Examples 14-16, the numbers of target organisms on host plants were assessed before treatment and after treatment. Assessment of mortality, growth stage effects, any other sub lethal effects, and phytotoxicity were carried out over a sampling period that could include any of 1 day after application (DAA), 3 DAA, 4 DAA, 7 DAA, 14 DAA, or 22 DAA for field experiments described in Examples 14-16.

For purposes of determining the dose or rate of ingredient being applied, concentrations of BUGOIL® were expressed as volume dilutions of milliliters (mL) of BUGOIL® per liter (L) of test solution, or mL/L. The BUGOIL® formulation contained canola oil (93.799% w/w) as a carrier oil, Tween20® (5.000% w/w) as an emulsifer, tagettes oil (0.600% w.w) as an active substance, thyme oil (0.600% w/w) as an active substance, and wintergreen oil (0.0001% w/w) as a synergizer (Plant Impact plc., Bamber Bridge, Preston, UK). In Examples 1-13, the dose or rate of active ingredients abamectin, imidacloprid, indoxacard, lambda-cyhalothrin, and spinosad, which were supplied in commercially available formulations under the product names listed below, was expressed as a dose or rate of grams of active ingredient (a.i.) applied per hectare (g (a.i.)/ha). In Examples 14-16, the dose or rate of active ingredients abamectin, imidacloprid, and lambda-cyhalothrin was expressed as a proportion of the recommended rate (expressed as “label dose” or “normal” or “N”) for the commercially available formulation being used for the plant/pest combination disclosed in the example, such that doses are expressed as “N” (label dose, not diluted) and dilutions are variously expressed, e.g., a 1:1 dilution or ½ normal strength as “N/2” or “N/2 dilution” or “½N” and likewise for N/6 dilution (⅙ normal strength), N/20 dilution ( 1/20 normal strength), usw.

Crop safety was evaluated for BUGOIL® alone and in mixture with the various acaricides/insecticides as described below. Very little phytoxocity was observed during the studies described below.

### Example 1

### Activity of Abamectin and BUGOIL® Against the Red Spider Mite, Tetranychus urticae

The activity of abamectin (formulated as VERTAN® 1.8 EC, active ingredient content 1.8% abamectin (80% abamectin B₁ₐ and 20% B_{1b}), Laboratorios Alcotán S.A., Dos Hermanas (Sevilla), Spain, Batch No. 10803007, April 2008) and BUGOIL® against the red spider mite, Tetranychus urticae was measured as described below. Test arenas consisted of excised leaf discs of the host plant Phaseolus vulgaris infested with the target organism, Tetranychus urticae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and abamectin) would result in mortality of between 25% (LD₂₅) and 50% (LD₅₀) for T. urticae. Rates corresponding to circa LD₂₅ (ca. LD₂₅) and circa LD₅₀ (ca. LD₅₀) were determined from dose-response curves showing the effects of a range of rates on survival of T. urticae on infested test arenas at 72 hours after application.

BUGOIL® was active against T. urticae when applied at rates above 2.5 mL/L, with the ca. LD₂₅ at around 5 mL/L and the ca. LD₅₀ at around 10 mL/L (see Table I).

Abamectin was active against T. urticae, as shown in the results of the range-finding assay for abamectin illustrated in FIG. 1A. FIG. 1A shows a dose-response curve for application of abamectin at rates of between 0.01 to 0.1 grams abamectin per hectare, expressed as grams active ingredient per hectare (g (a.i.)/ha) to test arenas infested with T. urticae, measured as percent (%) survival of T. urticae at 72 hours after application. The 75% survival rate (25% mortality) is indicated by a dashed line. FIG. 1A illustrates that the ca. LD₂₅ rate for abamectin against T. urticae was 0.025 g (a.i.)/ha, and the ca. LD₅₀ rate for abamectin against T. urticae was 0.040 g (a.i.)/ha.

Once the rates corresponding to ca. LD₂₅ and ca. LD₅₀ were established for BUGOIL® and for abamectin, further studies were undertaken to test the effects of varying rates of abamectin in mixtures with a fixed rate of BUGOIL®, and the effects of varying rates of BUGOIL® in mixtures with a fixed rate of abamectin, on the mortality (%) of T. urticae on infested test arenas at 72 hours after application.

FIG. 1B illustrates the effects of BUGOIL® at ca. LD₂₅ (5 mL/L BUGOIL®) in mixtures with abamectin at rates of between ca. LD₂₅ (0.025 g (a.i.)/ha) and ca. LD₅₀ (0.040 g (a.i.)/ha), and the effects of abamectin at ca. LD₂₅ (0.025 g (a.i.)/ha) in mixtures with BUGOIL® at rates of ca. LD₂₅ (5 mL/L), 8 mL/L, and ca. LD₅₀ (10 mL/L). Control treatments included a negative control corresponding to no treatment with either component, application of BUGOIL® at 5 ml/L corresponding to ca. LD₂₅ for BUGOIL®, and application of abamectin at 0.25 g(a.i.)/ha corresponding to ca. LD₂₅ for abamectin. Use of components in the range between ca. LD₂₅ and ca. LD₅₀ allowed comparison of the effects of each component alone and in combination, and allowed addition of predicted activities of mixtures for purposes of comparison with measured activities of the mixtures.

As illustrated in FIG. 1B, the pesticidal effects of BUGOIL® and abamectin mixtures were higher than the predicted additive pesticidal activity of each component in the mixture. The predicted (theoretical) activity of a mixture containing BUGOIL® at ca. LD₂₅ and abamectin at ca. LD₂₅ would be expected to be 50% at best (FIG. 1B, treatments 2 and 6), yet the observed pesticidal activity of the mixture of BUGOIL® at ca. LD₂₅ and abamectin at ca. LD₂₅ was nearly 70%. Thus, mixtures of BUGOIL® and abamectin showed synergistic pesticidal effects as an acaricide.

### Example 2

### Activity of Imidacloprid and BUGOIL® Against the Red Spider Mite, Tetranychus urticae

The activity of imidacloprid (formulated as CONFIDOR® 20 LS, active ingredient content 20% imidacloprid, Bayer Crop Science, Spain, Batch No. EQ6000353/5, November 2007) and BUGOIL® against the red spider mite, Tetranychus urticae was measured as described below. Test arenas consisted of excised leaf discs of the host plant Phaseolus vulgaris infested with the target organism, Tetranychus urticae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and imidacloprid) would result in mortality of between 25% (LD₂₅) and 50% (LD₅₀) for T. urticae. Rates corresponding to circa LD₂₅ (ca. LD₂₅) and circa LD₅₀ (ca. LD₅₀) were determined from dose-response curves showing the effects of a range of rates on survival of T. urticae on infested test arenas at 72 hours after application.

BUGOIL® was active against T. urticae when applied at rates above 2.5 milliliters/liter (mL/L), with the ca. LD₂₅ at around 5 mL/L and the ca. LD₅₀ at around 10 mL/L (see Table I).

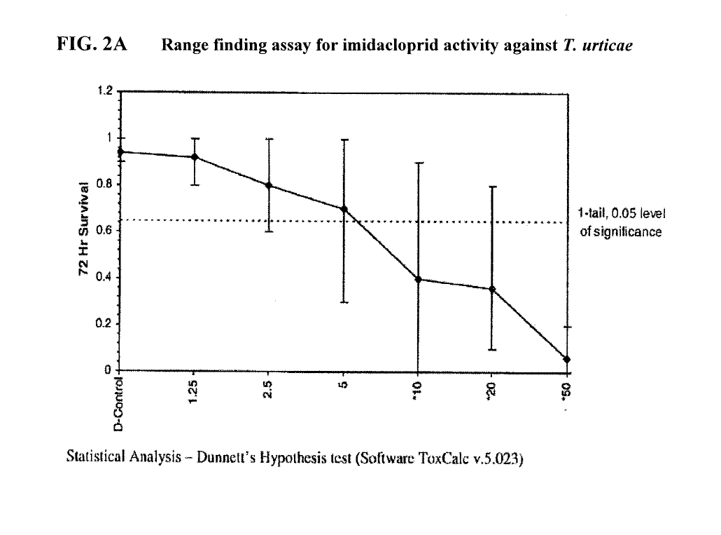

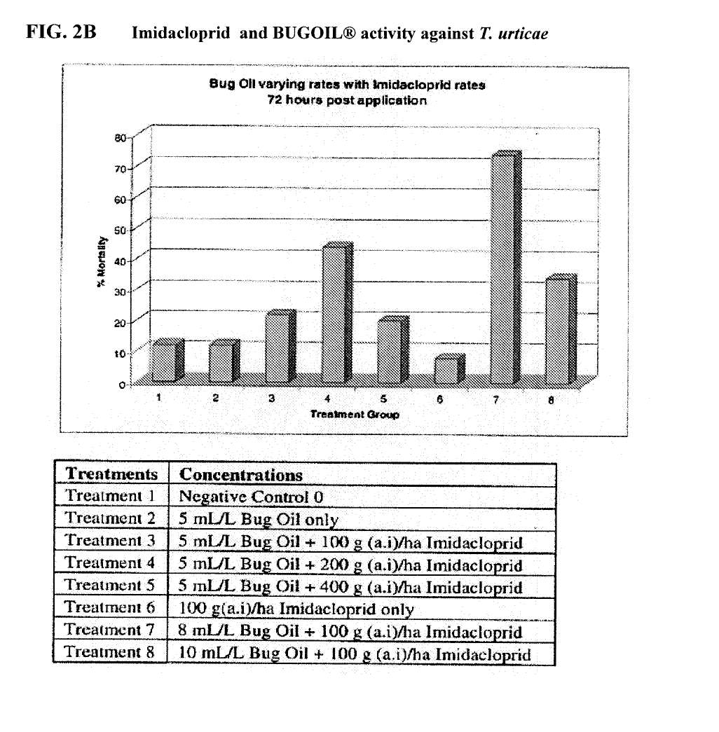

Results of the range-finding assay for imidacloprid are illustrated in FIG. 2A. FIG. 2A shows a dose-response curve for application of imidacloprid at rates of between 1.25 to 500 grams imidacloprid per hectare (g (a.i.)/ha) to test arenas infested with T. urticae, measured as percent (%) survival of T. urticae at 72 hours after application. The 75% survival rate (25% mortality) is indicated by a dashed line. The ca. LD₂₅ rate determined for imidacloprid against T. urticae was 100 g (a.i.)/ha, and the ca. LD₅₀ rate for imidacloprid against T. urticae was 400 g (a.i.)/ha (Table I) The results of the range finding bioassay for imidacloprid in FIG. 2A showed minimal activity against T. urticae at a rate as high as 400 g a.i./ha, with high variability in mortality rates, which is not unexpected as imidacloprid has not been reported effective against the red spider mite in the literature.

Once the rates corresponding to ca. LD₂₅ and ca. LD₅₀ were established for BUGOIL® and for imidacloprid, further studies were undertaken to test the effects of varying rates of abamectin in mixtures with a fixed rate of BUGOIL®, and the effects of varying rates of BUGOIL® in mixtures with a fixed rate of imidacloprid, on the mortality (%) of T. urticae on infested test arenas at 72 hours after application.

FIG. 2B illustrates the effects of BUGOIL® at ca. LD₂₅ (5 mL/L BUGOIL®) in mixtures with imidacloprid at rates of between ca. LD₂₅ (100 g (a.i.)/ha) and ca. LD₅₀ (400 g (a.i.)/ha), and the effects of imidacloprid at ca. LD₂₅ (100 g (a.i.)/ha) in mixtures with BUGOIL® at rates of ca. LD₂₅ (5 mL/L), 8 mL/L, and ca. LD₅₀ (10 mL/L). Control treatments included a negative control corresponding to no treatment with either component, application of BUGOIL® at 5 ml/L corresponding to ca.LD₂₅ for BUGOIL®, and application of imidacloprid only at 100 g(a.i.)/ha corresponding to ca. LD₂₅ for imidacloprid.

As illustrated in FIG. 2B, the pesticidal effects of BUGOIL® and imidacloprid mixtures showed a mortality level in some of the treatments that could be described as higher than the predicted outcomes. However, the results are not clear-cut, and in view of the high imidacloprid rate used, the conclusion was reached that it is unlikely that any useful effect was demonstrated. There is also some evidence that imidacloprid can increase the fecundity of the red spider mite at concentrations used to combat the target pests in this study and for this reason, such that it may be postulated that any further development of the concept of mixing BUGOIL® with this compound is not particularly advised.

### Example 3

### Activity of Indoxacarb and BUGOIL® Against the Red Spider Mite, Tetranychus urticae

The activity of indoxacarb (formulated as STEWARD® 30 WG, active ingredient content 30% indoxacarb, Dupont, Batch No. SEP08 CE 161, September 2008) and BUGOIL® against the red spider mite, Tetranychus urticae was measured as described below. Test arenas consisted of excised leaf discs of the host plant Phaseolus vulgaris infested with the target organism, Tetranychus urticae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and indoxacarb) would result in mortality of between 25% (LD₂₅) and 50% (LD₅₀) for T. urticae. Rates corresponding to circa LD₂₅ (ca. LD₂₅) and circa LD₅₀ (ca. LD₅₀) were determined from dose-response curves showing the effects of a range of rates on survival of T. urticae on infested test arenas at 72 hours after application.

BUGOIL® was active against T. urticae when applied at rates above 2.5 milliliters/liter (mL/L), with the ca. LD₂₅ at around 5 mL/L and the ca. LD₅₀ at around 10 mL/L (see Table I).

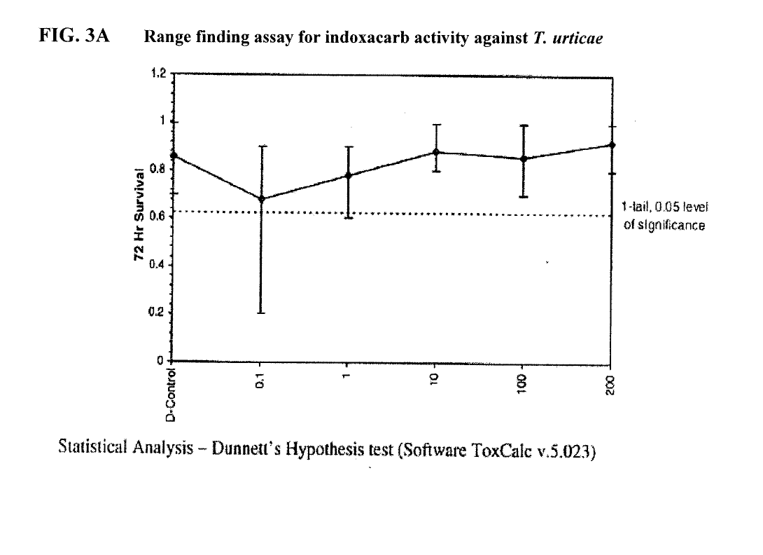

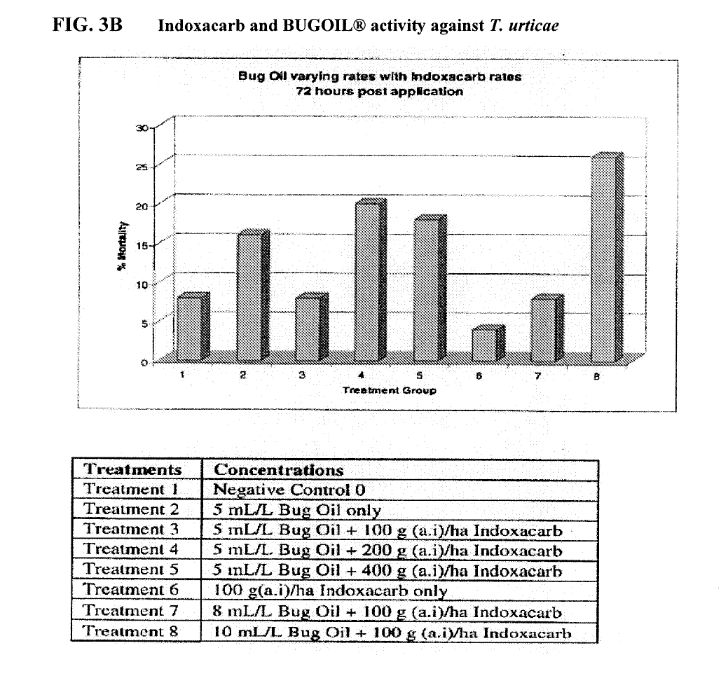

Results of the range-finding assay for indoxacarb are illustrated in FIG. 3A. FIG. 3A shows a dose-response curve for application of indoxacarb at rates of between 0.1 to 200 grams indoxacarb per hectare (g (a.i.)/ha) to test arenas infested with T. urticae, measured as percent (%) survival of T. urticae at 72 hours after application. The 75% survival rate (25% mortality) is indicated by a dashed line. The range-finding assay included tests for indoxacarb at rates of up to 400 g (a.i.)/ha (Table I). The ca. LD₂₅ rate determined for indoxacarb against T. urticae was 100 g (a.i.)/ha, and the ca. LD₅₀ rate for indoxacarb against T. urticae was 400 g (a.i.)/ha (Table I). Indoxocarb is a novel oxadiazine pesticide that has not been reported to have any specific toxicity to T. urticae, such that the results of the range finding bioassay are consistent with indoxocarb having minimal activity against T. urticae at a rate as high as 400 g a.i./ha, with high variability in mortality rates.

Once the rates corresponding to ca. LD₂₅ and ca. LD₅₀ were established for BUGOIL® and for indoxacarb, further studies were undertaken to test the effects of varying rates of indoxacarb in mixtures with a fixed rate of BUGOIL®, and the effects of varying rates of BUGOIL® in mixtures with a fixed rate of indoxacarb, on the mortality (%) of T. urticae on infested test arenas at 72 hours after application.

FIG. 3B illustrates the effects of BUGOIL® at ca. LD₂₅ (5 mL/L BUGOIL®) in mixtures with indoxacarb at rates of between ca. LD₂₅ (100 g (a.i.)/ha) and ca. LD₅₀ (400 g (a.i.)/ha), and the effects of indoxacarb at ca. LD₂₅ (100 g (a.i.)/ha) in mixtures with BUGOIL® at rates of ca. LD₂₅ (5 mL/L), 8 mL/L, and ca. LD₅₀ (10 mL/L). Control treatments included a negative control corresponding to no treatment with either component application of BUGOIL® at 5 ml/L corresponding to ca.LD₂₅ for BUGOIL®, and application of indoxacarb only at 100 g(a.i.)/ha corresponding to ca. LD₂₅ for indoxacarb.

As illustrated in FIG. 3B, indoxacarb did not perform especially well in mixtures with BUGOIL®, and observed mortality rates were lower than expected. Indoxacarb was not shown to be active against T. urticae when applied alone and did not demonstrate added potency in mixtures with BUGOIL®.

### Example 4

### Activity of Lambda-cyhalothrin and BUGOIL® Against the Red Spider Mite, Tetranychus urticae

The activity of lambda-cyhalothrin (formulated as KARATE KING® 2.5 WG, active ingredient content lambda-cyhalothrin 2.5%, Syngenta Crop Protection, Batch No. SOL7E10, July 2007) and BUGOIL® against the red spider mite, Tetranychus urticae was measured as described below. Test arenas consisted of excised leaf discs of the host plant Phaseolus vulgaris infested with the target organism, Tetranychus urticae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and lambda-cyhalothrin) would result in mortality of between 25% (LD₂₅) and 50% (LD₅₀) for T. urticae. Rates corresponding to circa LD₂₅ (ca. LD₂₅) and circa LD₅₀ (ca. LD₅₀) were determined from dose-response curves showing the effects of a range of rates on survival of T. urticae on infested test arenas at 72 hours after application.

BUGOIL® was active against T. urticae when applied at rates above 2.5 milliliters/liter (mL/L), with the ca. LD₂₅ at around 5 mL/L and the ca. LD₅₀ at around 10 mL/L (see Table I).

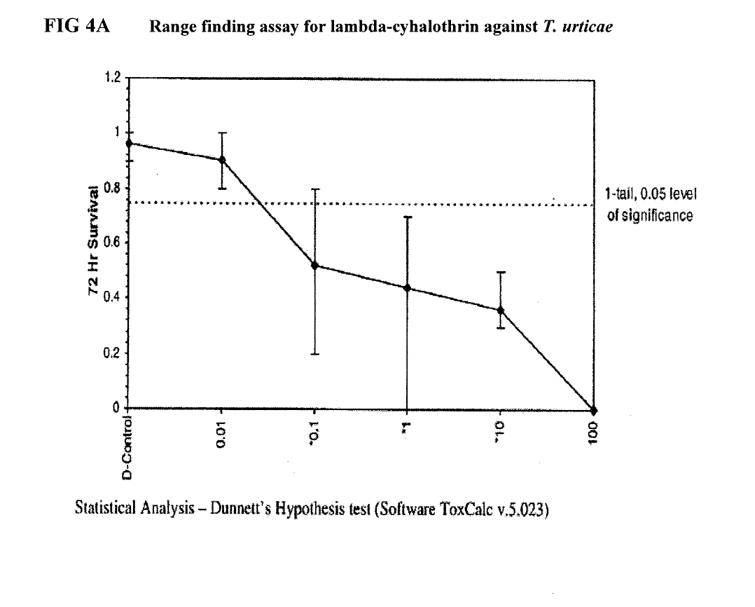

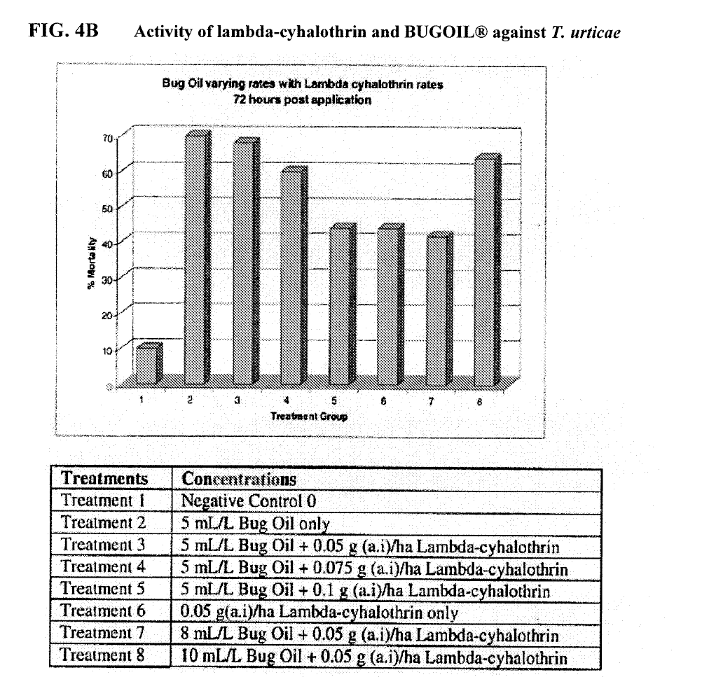

Lambda-cyhalothrin was active against T. urticae, as shown in the results of the range-finding assay for lambda-cyhalothrin illustrated in FIG. 4A. FIG. 4A shows a dose-response curve for application of lambda-cyhalothrin at rates of between 0.01 to 100 grams lambda-cyhalothrin per hectare (g (a.i.)/ha) to test arenas infested with T. urticae, measured as percent (%) survival of T. urticae at 72 hours after application. The 75% survival rate (25% mortality) is indicated by a dashed line. FIG. 4A illustrates that the ca. LD₂₅ rate for lambda-cyhalothrin against T. urticae was 0.05 g (a.i.)/ha, and the ca. LD₅₀ rate for lambda-cyhalothrin abamectin against T. urticae was 0.1 g (a.i.)/ha. Lambda-cyhalothrin, one of the pyrethroid insecticides, is known for its acaricidal effects on adult mites and the results from the range finding study showed a high rate of mortality at the lower levels tested.

Once the rates corresponding to ca. LD₂₅ and ca. LD₅₀ were established for BUGOIL® and for lambda-cyhalothrin, further studies were undertaken to test the effects of varying rates of lambda-cyhalothrin in mixtures with a fixed rate of BUGOIL®, and the effects of varying rates of BUGOIL® in mixtures with a fixed rate of lambda-cyhalothrin, on the mortality (%) of T. urticae on infested test arenas at 72 hours after application.

FIG. 4B illustrates the effects of BUGOIL® at ca. LD₂₅ (5 mL/L BUGOIL®) in mixtures with lambda-cyhalothrin at rates of between ca. LD₂₅ (0.05 g (a.i.)/ha) and ca. LD₅₀ (0.1 g (a.i.)/ha), and the effects of lambda-cyhalothrin at ca. LD₂₅ (0.05 g (a.i.)/ha) in mixtures with BUGOIL® at rates of ca. LD₂₅ (5 mL/L), 8 mL/L, and ca. LD₅₀ (10 mL/L). Control treatments included a negative control corresponding to no treatment with either component, application of BUGOIL® at 5 ml/L corresponding to ca.LD₂₅ for BUGOIL®, and application of lambda-cyhalothrin only at 0.05 g(a.i.)/ha corresponding to ca. LD₂₅ for lambda-cyhalothrin.

As illustrated in FIG. 4B, the pesticidal effects of each component were such that it was not clear whether the mixtures had synergistic, or even additive, acaricidal effects. BUGOIL® at ca. LD₂₅ showed significant pesticidal activity against T. urticae (FIG. 4B, Treatment 2) and lambda-cyhalothrin at ca. LD₂₅ showed pesticidal activity against T. urticae (FIG. 4B, Treatment 6). Adding these activities would result in a predicted (theoretical) mortality rate greater than the maximum of 100% mortality. All the mixtures of BUGOIL® and lambda-cyhalothrin (FIG. 4B, Treatments 3, 4, 5, 7, and 8) had significant pestidical activity. These results are consistent with the fact that lambda-cyhalothrin, a pyrethroid insecticide, is known for its acaricidal effects on adult mites. Thus, although the mixtures had significant pesticidal activity against T. urticae, this study did not demonstrate synergistic effects of the mixtures when compared with the predicted (theoretical) activity resulting from the addition of BUGOIL® activity at LD₂₅ and lambda cyhalothrin at the same LD value.

### Example 5

### Activity of Spinosad and BUGOIL® Against the Red Spider Mite, Tetranychus urticae

The activity of spinosad (formulated as SPINTOR® 48 SC, active ingredient content 48% spinosad, Dow AgroSciences, Batch No. VF0927036, November 2007) and BUGOIL® against the red spider mite, Tetranychus urticae was measured as described below. Test arenas consisted of excised leaf discs of the host plant Phaseolus vulgaris infested with the target organism, Tetranychus urticae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and spinosad) would result in mortality of between 25% (LD₂₅) and 50% (LD₅₀) for T. urticae. Rates corresponding to circa LD₂₅ (ca. LD₂₅) and circa LD₅₀ (ca. LD₅₀) were determined from dose-response curves showing the effects of a range of rates on survival of T. urticae on infested test arenas at 72 hours after application.

BUGOIL® was active against T. urticae when applied at rates above 2.5 milliliters/liter (mL/L), with the ca. LD₂₅ at around 5 mL/L and the ca. LD₅₀ at around 10 mL/L (see Table I).

Results of the range-finding assay for spinosad are illustrated in FIG. 5A. FIG. 5A shows a dose-response curve for application of spinosad at rates of between 0.1 to 100 grams spinosad per hectare (g (a.i.)/ha) to test arenas infested with T. urticae, measured as percent (%) survival of T. urticae at 72 hours after application. The ca. LD₂₅ rate determined for spinosad against T. urticae was 10 g (a.i.)/ha, and the ca. LD₅₀ rate for spinosad against T. urticae was 50 g (a.i.)/ha (Table I). Although the LD₂₅ and LD₅₀ rates for spinosad could be considered high when compared with some newly developed acaricides, a dose response to spinosad was observed over the range tested.

Once the rates corresponding to ca. LD₂₅ and ca. LD₅₀ were established for BUGOIL® and for spinosad, further studies were undertaken to test the effects of varying rates of spinosad in mixtures with a fixed rate of BUGOIL®, and the effects of varying rates of BUGOIL® in mixtures with a fixed rate of spinosad, on the mortality (%) of T. urticae on infested test arenas at 72 hours after application.

FIG. 5B illustrates the effects of BUGOIL® at ca. LD₂₅ (5 mL/L BUGOIL®) in mixtures with spinosad at rates of between ca. LD₂₅ (10 g (a.i.)/ha) and ca. LD₅₀ (50 g (a.i.)/ha), and the effects of spinosad at ca. LD₂₅ (10 g (a.i.)/ha) in mixtures with BUGOIL® at rates of ca. LD₂₅ (5 mL/L), 8 mL/L, and ca. LD₅₀ (10 mL/L). Control treatments included a negative control corresponding to no treatment with either component, application of BUGOIL® at 5 ml/L corresponding to ca.LD₂₅ for BUGOIL®, and application of spinosad only at 10 g(a.i.)/ha corresponding to ca. LD₂₅ for spinosad.

As illustrated in FIG. 5B, the pesticidal effects of BUGOIL® and spinosad mixtures were higher than the predicted additive pesticidal activity of each component in the mixture. The predicted (theoretical) activity of a mixture containing BUGOIL® at ca. LD₂₅ and spinosad at ca. LD₂₅ would be expected to be 50% at best (FIG. 5B, treatments 2 and 6), yet the observed pesticidal activity of the mixture of BUGOIL® at ca. LD₂₅ and spinosad at ca. LD₂₅ was approximately 80%, indicating synergistic effects. These synergistic effects were seen in all the results, when the spinosad or the BUGOIL® rate was increased. Specifically, mixtures of BUGOIL® at ca. LD₂₅ with spinosad at 15 g (a.i.)/ha and at 50 g (a.i)/ha (ca. LD₅₀) had mortality rates above 90%. Similarly, mixtures of spinosad at ca. LD₂₅ with higher rates of BUGOIL® had a mortality rate of around 70% for the mixture of spinosad at ca. LD₂₅ and BUGOIL® at 8 mL/L, and nearly 90% for the mixture of spinosad at ca. LD₂₅ and BUGOIL® at 10 mL/L (ca. LD₅₀). Although enhanced effects of mixtures could have been expected in view of the activity of each component, the increased pesticidal activity of the mixtures is significantly higher than the predicted (theoretical) activity of mixtures. Thus, mixtures of BUGOIL® and spinosad showed synergistic pesticidal effects as an acaricide.

### Example 6

### Activity of Abamectin and BUGOIL® Against the Green Peach Aphid, Myzus persicae

The activity of abamectin (formulated as VERTAN® 1.8 EC, active ingredient content 1.8% abamectin (80% abamectin B₁ₐ and 20% B_{1b}), Laboratorios Alcotán S.A., Dos Hermanas (Sevilla), Spain, Batch No. 10803007, April 2008)) and BUGOIL® against the green peach aphid, Myzus persicae, was measured as described below. Test arenas consisted of excised leaf discs of the host plant Brassica rapa chinensis infested with the target organism, Myzus persicae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and abamectin) would result in mortality of between 25% (LD₂₅) and 50% (LD₅₀) for M. persicae. Rates corresponding to ca. LD₂₅ and ca. LD₅₀ were determined from dose-response curves showing the effects of a range of rates on survival of M. persicae on infested test arenas at 72 hours after application.

When tested against the green peach aphid, M. persicae, BUGOIL® did not show defined aphicidal activity. In order to explore additive or synergistic effects of mixtures of BUGOIL® and abamectin on pesticidal activity against M. persicae, the BUGOIL® ca. LD₂₅ was considered to be 10 mL/L and LD₅₀ was considered to be 50 mL/L (see Table I).

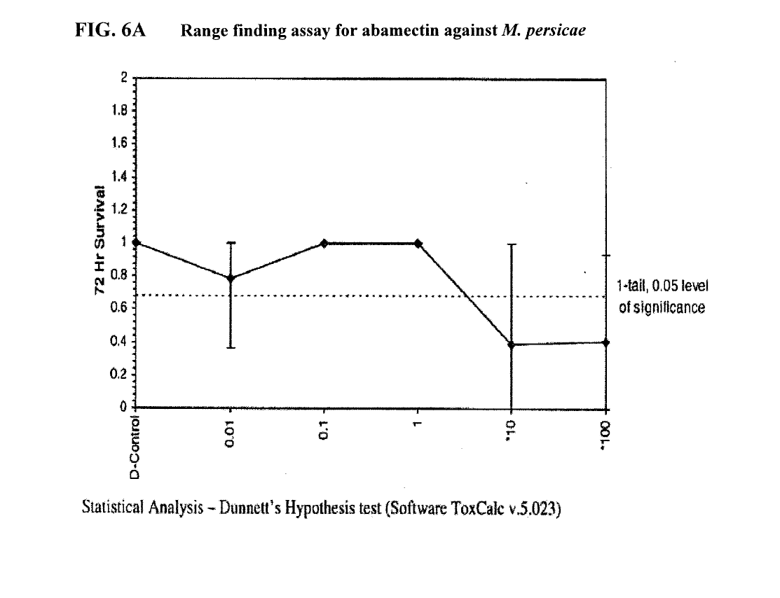

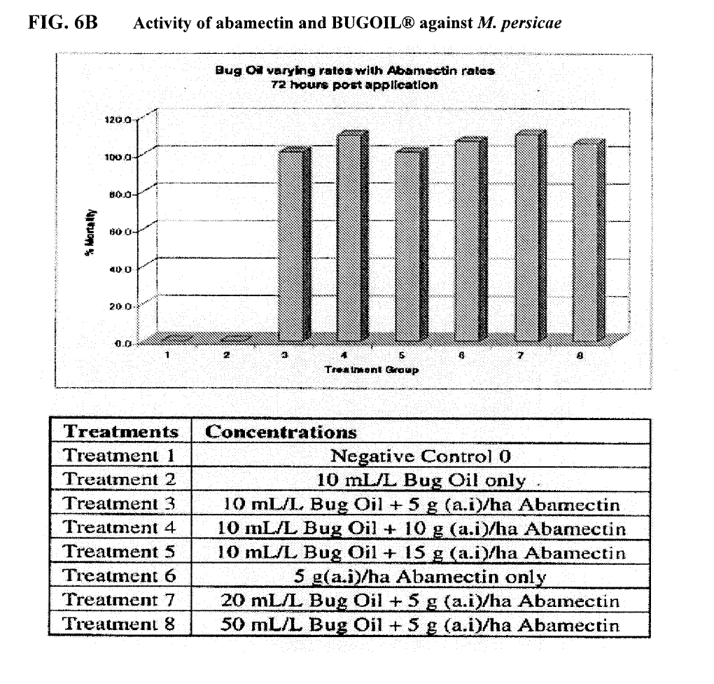

Abamectin was active against M. persicae, as shown in the results of the range-finding assay for abamectin illustrated in FIG. 6A. FIG. 6A shows a dose-response curve for application of abamectin at rates of between 0.1 to 100 grams abamectin per hectare (g (a.i.)/ha) to test arenas infested with M. persicae, measured as percent (%) survival of M. persicae at 72 hours after application. The 75% survival rate (25% mortality) is indicated by a dashed line. The ca. LD₂₅ rate for abamectin against M. persicae was determined to be 5 g (a.i.)/ha, and the ca. LD₅₀ rate was determined to be 15 g (a.i.)/ha (FIG. 6A, Table I).

Once the rates corresponding to ca. LD₂₅ and ca. LD₅₀ were established for BUGOIL® and for abamectin, further studies were undertaken to test the effects of varying rates of abamectin in mixtures with a fixed rate of BUGOIL®, and the effects of varying rates of BUGOIL® in mixtures with a fixed rate of abamectin, on the mortality (%) of M. persicae on infested test arenas at 72 hours after application.

FIG. 6B illustrates the effects of BUGOIL® at ca. LD₂₅ (10 mL/L BUGOIL®) in mixtures with abamectin at rates of ca. LD₂₅ (5 g (a.i.)/ha), 10 g (a.i)/ha, and ca. LD₅₀ (15 g (a.i.)/ha), and the effects of abamectin at ca. LD₂₅ (5 g (a.i.)/ha) in mixtures with BUGOIL® at rates of ca. LD₂₅ (10 mL/L), 20 mL/L, and ca. LD₅₀ (50 mL/L). Control treatments included a negative control corresponding to no treatment with either component application of BUGOIL® at 10 ml/L corresponding to ca.LD₂₅ for BUGOIL®, and application of abamectin at 5 g(a.i.)/ha corresponding to ca. LD₂₅ for abamectin.

As illustrated in FIG. 6B, BUGOIL® alone at ca. LD₂₅ had almost no pesticidal effect on M. persicae (FIG. 6B, Treatment 2) and all treatments including abamectin resulted in mortality rates near 100% (FIG. 6B, Treatments 3-8). In this study, the effects of abamectin at LD₂₅ and LD₅₀ levels were not easy to reproduce against M. persicae adults. Therefore, although the leaf disc infestation technique using adult aphids showed high efficacy of abamectin applied as VERTAN® at LD₂₅ (5 g (a.i.)/ha) or above, with a mortality rate of nearly 100%, the data was inconclusive with respect to determining whether a mixture of abamectin with BUGOIL® demonstrated synergistic effects.

### Example 7

### Activity of Imidacloprid and BUGOIL® Against the Green Peach Aphid, Myzus persicae

The activity of imidacloprid (formulated as CONFIDOR® 20 LS, active ingredient content 20% imidacloprid, Bayer Crop Science, Spain, Batch No. EQ6000353/5, November 2007) and BUGOIL® against the green peach aphid, Myzus persicae, was measured as described below. Test arenas consisted of excised leaf discs of the host plant Brassica rapa chinensis infested with the target organism, Myzus persicae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and imidacloprid) would result in mortality of between 25% (LD₂₅) and 50% (LD₅₀) for M. persicae. Rates corresponding to ca. LD₂₅ and ca. LD₅₀ were determined from dose-response curves showing the effects of a range of rates on survival of M. persicae on infested test arenas at 72 hours after application.

When tested against the green peach aphid, M. persicae, BUGOIL® did not show defined aphicidal activity. In order to explore additive or synergistic effects of mixtures of BUGOIL® and imidacloprid on pesticidal activity against M. persicae, the BUGOIL® ca. LD₂₅ was considered to be 10 mL/L and LD₅₀ was considered to be 50 mL/L (see Table I).

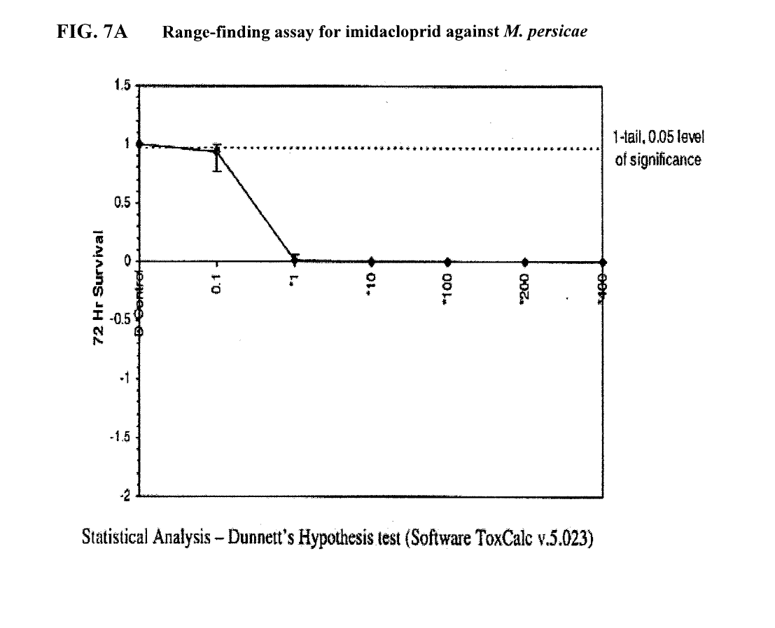

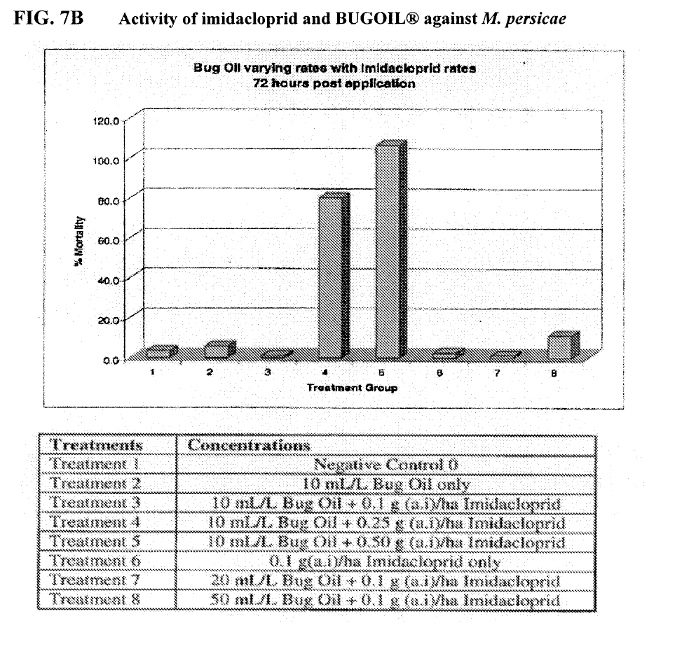

Imidacloprid was active against M. persicae, as shown in the results of the range-finding assay for imidacloprid illustrated in FIG. 7A. FIG. 7A shows a dose-response curve for application of imidacloprid at rates of between 0.1 to 400 grams imidacloprid per hectare (g (a.i.)/ha) to test arenas infested with M. persicae, measured as percent (%) survival of M. persicae at 72 hours after application. The ca. LD₂₅ rate for imidacloprid against M. persicae was determined to be 0.1 g (a.i.)/ha, and the ca. LD₅₀ rate was determined to be 0.5 g (a.i.)/ha (FIG. 7A, Table I). Imidacloprid is well documented as an effective insecticide against M. persicae and the present results are in agreement with the known activity of imidacloprid.

Once the rates corresponding to ca. LD₂₅ and ca. LD₅₀ were established for BUGOIL® and for imidacloprid, further studies were undertaken to test the effects of varying rates of imidacloprid in mixtures with a fixed rate of BUGOIL®, and the effects of varying rates of BUGOIL® in mixtures with a fixed rate of imidacloprid, on the mortality (%) of M. persicae on infested test arenas at 72 hours after application.

FIG. 7B illustrates the effects of BUGOIL® at ca. LD₂₅ (10 mL/L BUGOIL®) in mixtures with imidacloprid at rates of ca. LD₂₅ (0.1 g (a.i.)/ha), 0.25 g (a.i.)/ha, and ca. LD₅₀ (0.5 g (a.i.)/ha), and the effects of imidacloprid at ca. LD₂₅ (0.1 g (a.i.)/ha) in mixtures with BUGOIL® at rates of ca. LD₂₅ (10 mL/L), 20 mL/L, and ca. LD₅₀ (50 mL/L). Control treatments included a negative control corresponding to no treatment with either component, application of BUGOIL® at 10 ml/L (ca.LD₂₅) and application of imidacloprid at 0.1 g(a.i.)/ha (ca. LD₂₅).

As illustrated in FIG. 7B, neither BUGOIL® alone at ca.LD₂₅ (10 mL/L), nor imidacloprid alone at ca.LD₂₅ (0.1 g(a.i.)/ha), nor a mixture of BUGOIL® at ca.LD₂₅ and imidacloprid at ca.LD₂₅ had significant pesticidal activity against M. persicae in this study (FIG. 7B, Treatments 2, 3, and 6). However, BUGOIL® at ca.LD₂₅ (10 mL/L) and higher levels of imidacloprid showed very strong pesticidal activity against M. persicae, with mortality rates of nearly 80% for imidacloprid at 0.25 g (a.i.)/ha, and around 100% for 0.5 g (a.i.)/ha (ca. LD₅₀) (FIG. 7B, Treatments 4 and 5). Imidacloprid at ca. LD₂₅ (0.1 g (a.i.)/ha), with higher levels of BUGOIL® at 20 mL/L and 50 mL/L (ca. LD₅₀) did not show a significant effect on pesticidal activity against M. persicae.

Although imidacloprid is well documented as an effective insecticide against M. persicae, previous studies have demonstrated that contact activity (i.e., activity when the insect is contacted directly with imidacloprid) is superior to applying the insecticide to the plant and then placing the aphids on the plant. The results of the present study are produced by direct application to the pest and the leaf disc. As shown in FIG. 7A for the range finder assay, imidacloprid at concentrations of 1 g a.i./ha and above gave mortality rates of greater than 90% (LD₉₀). In order to obtain the LD₂₅ and LD₅₀ for midacloprid against M. persicae, the concentration range was lowered to cover the range 0.1 to 1 g a.i./ha, and an LD₂₅ rate of 0.1 g a.i./ha was chosen as the rate to be used in the mixture study. Although high mortality rates were seen in mixtures of the 0.25 and 0.5 g a.i./ha with 10 mL/L BUGOIL®, but the results for imidacloprid at 0.1 g a.i./ha were inconsistent with the initial results obtained on the range finder study. Therefore, a specific effect that could be attributed to the presence of BUGOIL® in a mixture of imidacloprid and BUGOIL® could not be identified.

### Example 8

### Activity of Indoxacarb and BUGOIL® Against the Green Peach Aphid, Myzus persicae

The activity of indoxacarb (formulated as STEWARD® 30 WG, active ingredient content 30% indoxacarb, Dupont, Batch No. SEP08 CE 161, September 2008) and BUGOIL® against the green peach aphid, Myzus persicae, was measured as described below. Test arenas consisted of excised leaf discs of the host plant Brassica rapa chinensis infested with the target organism, Myzus persicae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and indoxacarb) would result in mortality of between 25% (LD₂₅) and 50% (LD₅₀) for M. persicae. Rates corresponding to ca. LD₂₅ and ca. LD₅₀ were determined from dose-response curves showing the effects of a range of rates on survival of M. persicae on infested test arenas at 72 hours after application.

When tested against the green peach aphid, M. persicae, BUGOIL® did not show defined aphicidal activity. In order to explore additive or synergistic effects of mixtures of BUGOIL® and indoxacarb on pesticidal activity against M. persicae, the BUGOIL® ca. LD₂₅ was considered to be 10 mL/L and LD₅₀ was considered to be 50 mL/L (see Table I).

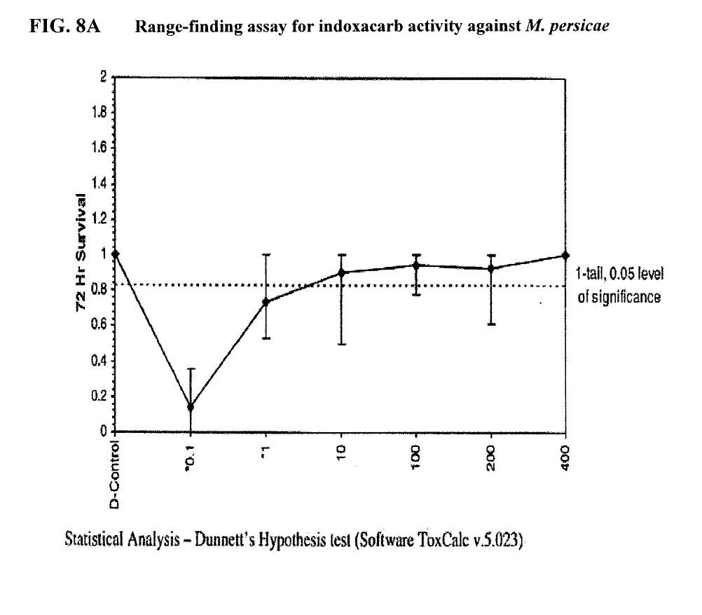

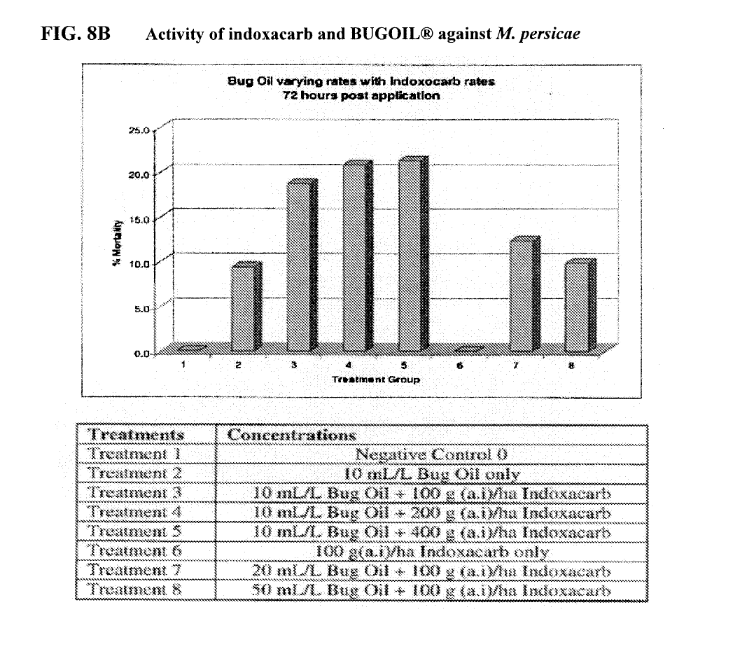

Indoxacarb was tested against M. persicae over a range of concentrations, similar to those used on the spider mite, and little aphicidal activity was detected across the test range, as shown in the results of the range-finding assay for indoxacarb illustrated in FIG. 8A, where indoxacarb at rates of between 0.1 to 400 grams indoxacarb per hectare (g (a.i.)/ha) to test arenas infested with M. persicae, measured as percent (%) survival of M. persicae at 72 hours after application. Although there appeared to be a slight increase in mortality rates over the control, statistical analysis showed no significance. These results were not unexpected as Lepidoptera, certain Homoptera and Coleoptera are the known targets for indoxacarb, and it is reported that indoxacarb is not particularly effective against aphids, although there may be a longer term effect on some sucking insects. Nonetheless, the ca. LD₂₅ rate for indoxacarb against M. persicae was considered to be 100 g (a.i.)/ha, and the ca. LD₅₀ rate was considered to be 400 g (a.i.)/ha (FIG. 8A, Table I)

Once the rates corresponding to ca. LD₂₅ and ca. LD₅₀ were established for BUGOIL® and for indoxacarb, further studies were undertaken to test the effects of varying rates of indoxacarb in mixtures with a fixed rate of BUGOIL®, and the effects of varying rates of BUGOIL® in mixtures with a fixed rate of indoxacarb, on the mortality (%) of M. persicae on infested test arenas at 72 hours after application.

FIG. 8B illustrates the effects of BUGOIL® at ca. LD₂₅ (10 mL/L BUGOIL®) in mixtures with indoxacarb at rates of ca. LD₂₅ (100 g (a.i.)/ha), 200 g (a.i.)/ha, and ca. LD₅₀ (400 g (a.i.)/ha), and the effects of indoxacarb at ca. LD₂₅ (100 g (a.i.)/ha) in mixtures with BUGOIL® at rates of ca. LD₂₅ (10 mL/L), 20 mL/L, and ca. LD₅₀ (50 mL/L). Control treatments included a negative control corresponding to no treatment with either component, application of BUGOIL® at 10 ml/L (ca.LD₂₅) and application of indoxacarb at 100 g(a.i.)/ha (ca. LD₂₅).

As illustrated in FIG. 8B, indoxacarb alone at 100 g (a.i.)/ha did not have measurable pesticidal activity against M. persicae on infested test arenas by 72 hours after application (FIG. 8B, Treatment 6). BUGOIL® alone at 10 mL/L (ca. LD₂₅) achieved mortality of nearly 10% (FIG. 8B, Treatment 2). Mixtures of BUGOIL® at 10 mL/L with indoxacarb at 100 g (a.i.)/ha, 200 g (a.i.)/ha, and 400 g (a.i.)/ha, achieved mortality rates of nearly 20% (FIG. 8B, Treatments 3, 4, and 5). Mixtures indoxacarb at 100 g (a.i.)/ha and BUGOIL® at 20 mL/L and 50 mL/L achieved mortality rates of around 10% (FIG. 8B, Treatments 7 and 8), not significantly different from the results of BUGOIL® alone at 10 mL/L. As noted above, the low aphicidal activity observed for indoxacarb against M. persicae was not unexpected as indoxacarb is generally used against Lepidoptera, certain Homoptera and Coleoptera and is reportedly not particularly effective against aphids. Although mixtures of BUGOIL® with higher rates of indoxacarb showed increased pesticidal activity compared with BUGOIL® alone or indoxacarb alone, the mortality level remained below 25%.

### Example 9

### Activity of Lambda-Cyhalothrin and BUGOIL® Against the Green Peach Aphid, Myzus persicae

The activity of lambda-cyhalothrin (formulated as KARATE KING® 2.5 WG, active ingredient content lambda-cyhalothrin 2.5%, Syngenta Crop Protection, Batch No. SOL7E10, July 2007)) and BUGOIL® against the green peach aphid, Myzus persicae, was measured as described below. Test arenas consisted of excised leaf discs of the host plant Brassica rapa chinensis infested with the target organism, Myzus persicae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and lambda-cyhalothrin) would result in mortality of between 25% (LD₂₅) and 50% (LD₅₀) for M. persicae. Rates corresponding to ca. LD₂₅ and ca. LD₅₀ were determined from dose-response curves showing the effects of a range of rates on survival of M. persicae on infested test arenas at 72 hours after application.

When tested against the green peach aphid, M. persicae, BUGOIL® did not show defined aphicidal activity. In order to explore additive or synergistic effects of mixtures of BUGOIL® and lambda-cyhalothrin on pesticidal activity against M. persicae, the BUGOIL® ca. LD₂₅ was considered to be 10 mL/L and LD₅₀ was considered to be 50 mL/L (see Table I).

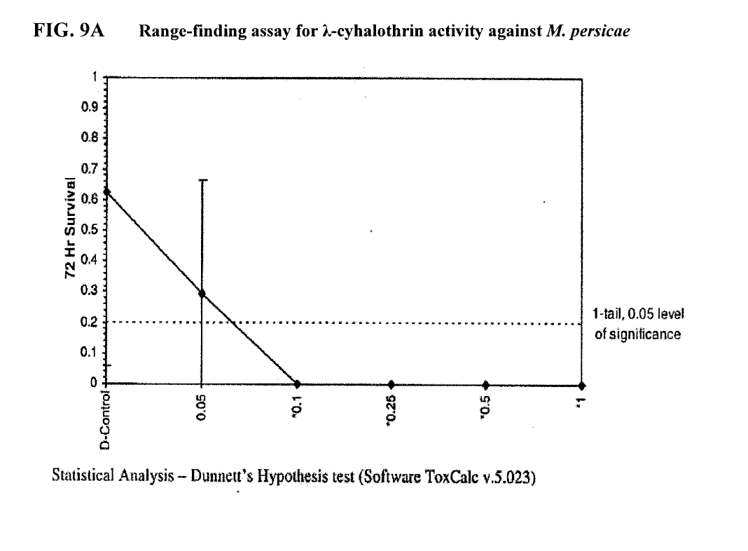

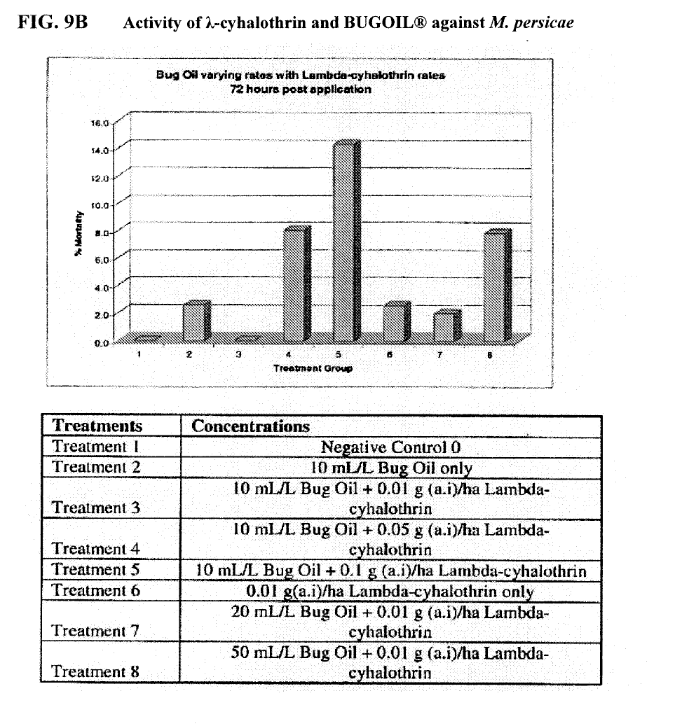

Lambda-cyhalothrin was shown to have high aphicidal activity, as shown in the results of the range-finding assay illustrated in FIG. 9A. As shown in FIG. 9A, survival rates of lambda-cyhalothrin applied at rates of between 0.05 and 1 g lambda-cyhalothrin per hectare (g (a.i.)/ha) to test arenas infested with M. persicae, were measured as percent (%) survival of M. persicae at 72 hours after application. The ca. LD₂₅ rate for lambda-cyhalothrin against M. persicae was determined to be 0.01 g (a.i.)/ha, and the ca. LD₅₀ rate was determined to be 0.01 g (a.i.)/ha (FIG. 9A, Table I).

Once the rates corresponding to ca. LD₂₅ and ca. LD₅₀ were established for BUGOIL® and for lambda-cyhalothrin, further studies were undertaken to test the effects of varying rates of lambda-cyhalothrin in mixtures with a fixed rate of BUGOIL®, and the effects of varying rates of BUGOIL® in mixtures with a fixed rate of lambda-cyhalothrin, on the mortality (%) of M. persicae on infested test arenas at 72 hours after application.

FIG. 9B illustrates the effects of BUGOIL® at ca. LD₂₅ (10 mL/L BUGOIL®) in mixtures with lambda-cyhalothrin at rates of ca. LD₂₅ (0.01 g (a.i.)/ha), 0.05 g (a.i.)/ha, and ca. LD₅₀ (0.1 g (a.i.)/ha), and the effects of lambda-cyhalothrin at ca. LD₂₅ (0.01 g (a.i.)/ha) in mixtures with BUGOIL® at rates of ca. LD₂₅ (10 mL/L), 20 mL/L, and ca. LD₅₀ (50 mL/L). Control treatments included a negative control corresponding to no treatment with either component, application of BUGOIL® at 10 ml/L (ca.LD₂₅) and application of lambda-cyhalothrin at 0.01 g(a.i.)/ha (ca. LD₂₅).

As illustrated in FIG. 9B, mixtures of BUGOIL® at a rate of 10 mL/L, with lambda-cyhalothrin at 0.05 or 0.1 g (a.i.)/ha, achieved higher mortality rates that BUGOIL® alone at 10 mL/L, or lambda-cyhalothrin at 0.01 g (a.i.)/ha, or a mixture of BUGOIL® at 10 mL/L and lambda-cyhalothrin at 0.01 g (a.i.)/ha. A mixture of lambda-cyhalothrin at 0.01 g (a.i.)/ha and BUGOIL® at 50 mL/L also showed achieved a higher mortality rate. However, none of these treatments achieve high mortality rates, and a consistent pattern of synergistic effects was observed.

### Example 10

### Activity of Spinosad and BUGOIL® Against the Green Peach Aphid, Myzus persicae

The activity of spinosad (formulated as SPINTOR® 48 SC, active ingredient content 48% spinosad, Dow AgroSciences, Batch No. VF0927036, November 2007) and BUGOIL® against the green peach aphid, Myzus persicae, was measured as described below. Test arenas consisted of excised leaf discs of the host plant Brassica rapa chinensis infested with the target organism, Myzus persicae. A track sprayer was used to spray the test solutions at an application volume of 200 L/ha. Assessment of mortality (survival), any other sub lethal effects and phytotoxicity were carried out at 24 hours and 72 hours post application (3DAA).

Range-finding assays were carried our to identify the rate (dose) at which each component (BUGOIL® and spinosad) would result in mortality of between 25% (LD₂₅) and 50% (LD₅₀) for M. persicae. Rates corresponding to ca. LD₂₅ and ca. LD₅₀ were determined from dose-response curves showing the effects of a range of rates on survival of M. persicae on infested test arenas at 72 hours after application.

When tested against the green peach aphid, M. persicae, BUGOIL® did not show defined aphicidal activity. In order to explore additive or synergistic effects of mixtures of BUGOIL® and spinosad on pesticidal activity against M. persicae, the BUGOIL® ca. LD₂₅ was considered to be 10 mL/L and LD₅₀ was considered to be 50 mL/L (see Table I).

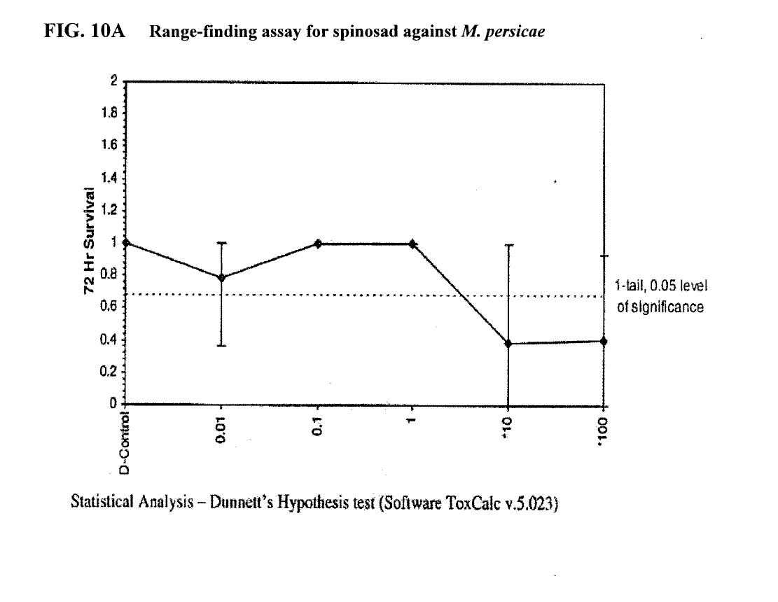

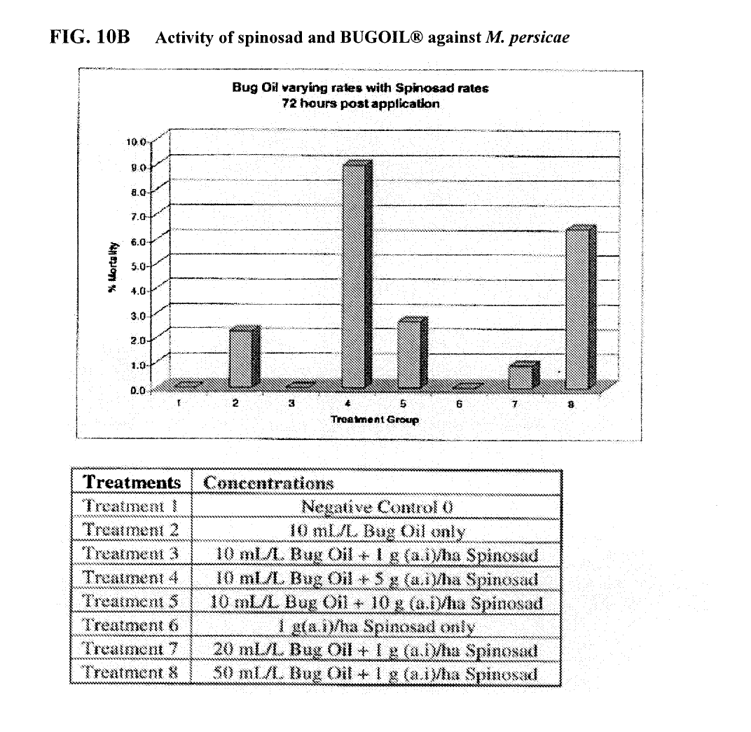

Spinosad showed variable aphicidal activity across the test range, as shown in the results of the range-finding assay for spinosad illustrated in FIG. 10A. As shown in FIG. 10A, spinosad was applied at rates of between 0.01 to 100 spinosad per hectare (g (a.i.)/ha) to test arenas infested with M. persicae, and activity was measured as percent (%) survival of M. persicae at 72 hours after application. Although the literature contains reports that spinosad may have efficacy against the green peach aphid, the results of many tests in these reports are variable. The ca. LD₂₅ rate for spinosad against M. persicae was determined to be 10 g (a.i.)/ha, and the ca. LD₅₀ rate was determined to be 50 g (a.i.)/ha (FIG. 10A, Table I).

Once the rates corresponding to ca. LD₂₅ and ca. LD₅₀ were established for BUGOIL® and for spinosad, further studies were undertaken to test the effects of varying rates of spinosad in mixtures with a fixed rate of BUGOIL®, and the effects of varying rates of BUGOIL® in mixtures with a fixed rate of spinosad, on the mortality (%) of M. persicae on infested test arenas at 72 hours after application.

FIG. 10B illustrates the effects of BUGOIL® at ca. LD₂₅ (10 mL/L BUGOIL®) in mixtures with spinosad at rates of ca. LD₂₅ (1 g (a.i.)/ha), 5 g (a.i.)/ha, and ca. LD₅₀ (10 g (a.i.)/ha), and the effects of spinosad at ca. LD₂₅ (1 g (a.i.)/ha) in mixtures with BUGOIL® at rates of ca. LD₂₅ (10 mL/L), 20 mL/L, and ca. LD₅₀ (50 mL/L). Control treatments included a negative control corresponding to no treatment with either component, application of BUGOIL® at 10 ml/L (ca.LD₂₅) and application of spinosad lambda-cyhalothrin at 1 g (a.i.)/ha (ca. LD₂₅).

The results were variable and inconclusive. Spinosad alone at 1 g (a.i.)/ha, or a mixture of spinosad at 1 g (a.i.)/ha and BUGOIL® at 10 mL/L had no measurable pesticidal activity. BUGOIL® alone at 10 mL/L had a small pesticidal effect, and mixtures of BUGOIL® at 10 mL/L with spinosad at 5 g (a.i.)/ha or 10 g (a.i.)/has, and spinosad at 1 g (a.i.)/ha with BUGOIL® at 20 mL/L and 50 mL/L had small pesticidal effects. However, the mortality rates were low, not even 10%, and the results did not show a consistent pattern suggesting synergistic effects. As noted above, reports of spinosad having efficacy against the green peach aphid include variable test results.

### Example 11

### Summary of Range-Finding Bioassays for BUGOIL® and Pesticidal Formulations

Table 1 below presents a summary of the range-finding assays for BUGOIL® and for each formulation, against each test organism, as described in Examples 1-10 above and illustrated in FIGS. 1A-10A, and confirms the rates selected as ca. LD₂₅ and ca. LD₅₀ for use in the tests described in Examples 1-10 above and illustrated in FIGS. 1B-10B. With respect to BUGOIL® effects on the green peach aphid, Myzus persicae, it was understood that BUGOIL® showed little or no defined aphicidal activity, the LD₂₅ value of 10 mL/L and the LD₅₀ value of 50 mL/L listed in Table I, it was recognized that these rates are not necessarily the LD₂₅ and the LD₅₀ values, as there was little or no response over the dose range.

Examination of Table I will show that not all the active ingredients tested for each pest showed any particular potency against the chosen pest target. This did not preclude them from the studies described in Examples 1-10, as these studies were undertaken to investigate whether there was any potential for the mixtures of the products to produce an effect that may not have been indicated by the performance of the individual compound against the target organism.

TABLE I
Summary Table of Range-Finding Assays

| Range Finder | | | | | Concentration | | Concentration | |
|---|---|---|---|---|---|---|---|---|
| Bioassay | | | Concentration | | effect ca | | effect ca | |
| Study No. | Active (Product) | Target | Ranges tested | | LD₂₅ 3DAA | | LD₅₀ 3DAA | |
| CEMS-4232 | Bug Oil | T. urticae | 1.25 to 50 | mL/L | 5 | mL/L | 10 | mL/L |
| CEMS-4241 | Abamectin (Vertan) | T. urticae | 0.01 to 0.1 | g a.i./ha | 0.025 | g a.i./ha | 0.04 | g a.i./ha |
| CEMS-4242 | Imidacloprid (Condifor 20 | T. urticae | 0.01 to 400 | g a.i./ha | 100 | g a.i/ha* | 400 | g a.i./ha* |
| | LS) | | | | | | | |
| CEMS-4243 | Indoxacarb (Steward) | T. urticae | 0.01 to 400 | g a.i./ha | 100 | g a.i./ha* | 400 | g a.i./ha* |
| CEMS-4244 | Lambda-cyhalothrin | T. urticae | 0.01 to 100 | g a.i./ha | 0.05 | g a.i./ha | 0.1 | g a.i./ha |
| | (Karate King) | | | | | | | |
| CEMS-4245 | Spinosad (SpinTor) | T. urticae | 0.01 to 100 | g a.i./ha | 10 | g a.i./ha | 50 | g a.i./ha |
| CEMS-4264 | Bug Oil | M. persicae | 1.25 to 50 | mL/L | 10 | mL/L* | 50 | mL/L* |
| CEMS-4265 | Abamectin (Vertan) | M. persicae | 0.01 to 100 | g a.i./ha | 5 | g a.i./ha | 15 | g a.i./ha |
| CEMS-4266 | Imidacloprid (Condifor 20 | M. persicae | 0.1 to 1 | g a.i./ha | 0.1 | g a.i./ha | 0.5 | g a.i./ha |
| | LS) | | | | | | | |
| CEMS-4267 | Indoxacarb (Steward) | M. persicae | 0.1 to 400 | g a.i./ha | 100 | g a.i./ha* | 400 | g a.i./ha* |
| CEMS-4268 | Lambda-cyhalothrin | M. persicae | 0.05 to 1 | g a.i./ha | 0.01 | g a.i./ha | 0.1 | g a.i./ha |
| | (Karate King) | | | | | | | |
| CEMS-4269 | Spinosad (SpinTor) | M. persicae | 0.01 to 100 | g a.i./ha | 10 | g a.i./ha* | 50 | g a.i./ha* |

*These rates are not necessarily at the LD₂₅ and LD₅₀ levels as there was little or no response to the dose range.

### Example 12

### Summary of Effects of Pesticidal Mixtures on the Red Spider Mite, Tetranychus urticae

Table II below summarizes results of studies described in Examples 1-5 above, designed to explore the effects of varying rates of each formulation in mixtures (at rates from LD₂₅ and LD₅₀) with a fixed rate of BUGOIL®, and the effects of varying rates of BUGOIL® in mixtures (at rates from LD₂₅ and LD₅₀) with a fixed rate of each formulation, on the mortality (%) of T. urticae on infested test arenas at 72 hours after application. The mortality rates of mixtures were measured and compared with the theoretical (predicted) mortality rate of the mixture based on addition of the effects of each component of the mixture, where the percentage values reported in Table II were rounded to the next integer. Table II indicates the percentage increase in mortality rate over the theoretical (predicted) mortality rate for each mixture. Beneficial effects were seen with mixtures of BUGOIL® with abamectin, and BUGOIL® with spinosad against T. urticae in the present studies, indicating that the mixtures had synergistic pesticidal effects.

TABLE II
Summary of Effects on Two Spotted Red Spider Mite (T. urticae)

| Mixture | | | | Percentage | |
|---|---|---|---|---|---|
| Bioassay | | | Concentration | increase over | |
| Study | Product/Active | Target | Ranges tested | theoretical# | Comments |
| CEMS-4281 | Abamectin (Vertan) | T. urticae | 5 mL/L Bug Oil + | | Very clear |
| | | | 0.025 g a.i./ha | 50 | beneficial |
| | | | 0.035 g a.i./ha | 85 | effect |
| | | | 0.040 g a.i./ha | 85 | |
| CEMS-4281 | Abamectin (Vertan) | T. urticae | 0.025 g a.i./ha + | | Very clear |
| | | | 5 mL/L Bug Oil | 50 | beneficial |
| | | | 8 mL/L Bug Oil | 85 | effect |
| | | | 10 mL/L Bug Oil | 80 | |
| CEMS-4283 | Imidacloprid (Confidor 20 | T. urticae | 5 mL/L Bug Oil + | | No consistent |
| | LS) | | 100 g a.i./ha | <5 | effect |
| | | | 200 g a.i./ha | 20 | |
| | | | 400 g a.i./ha | <5 | |
| CEMS-4283 | Imidacloprid (Confidor 20 | T. urticae | 100 g a.i./ha + | | No consistent |
| | LS) | | 5 mL/L Bug Oil | <5 | effect |
| | | | 8 mL/L Bug Oil | 55 | |
| | | | 10 mL/L Bug Oil | 15 | |
| CEMS-4284 | Indoxacarb (Steward) | T. urticae | 5 mL/L Bug Oil + | | No effect |
| | | | 100 g a.i./ha | <5 | |
| | | | 200 g a.i./ha | <5 | |
| | | | 400 g a.i./ha | <5 | |
| CEMS-4284 | Indoxacarb (Steward) | T. urticae | 100 g a.i./ha + | | No effect |
| | | | 5 mL/L Bug Oil | <5 | |
| | | | 8 mL/L Bug Oil | <5 | |
| | | | 10 mL/L Bug Oil | 6 | |
| CEMS-4285 | Lambda-cyhalothrin | T. urticae | 5 mL/L Bug Oil + | | High rates of |
| | (Karate King) | | 0.05 g a.i./ha | 10 | mortality but |
| | | | 0.075 g a.i./ha | 3 | no significant |
| | | | 0.1 g a.i./ha | <5 | increase |
| CEMS-4285 | Lambda-cyhalothrin | T. urticae | 0.05 g a.i./ha + | | High rates of |
| | (Karate King) | | 5 mL/L Bug Oil | 10 | mortality but |
| | | | 8 mL/L Bug Oil | <5 | no significant |
| | | | 10 mL/L Bug Oil | 5 | increase |
| CEMS-4286 | Spinosad (SpinTor) | T. urticae | 5 mL/L Bug Oil + | | Beneficial |
| | | | 10 g a.i./ha | 60 | effect seen |
| | | | 15 g a.i./ha | 75 | |
| | | | 20 g a.i./ha | 75 | |
| CEMS-4286 | Spinosad (SpinTor) | T. urticae | 10 g a.i./ha + | | Beneficial |
| | | | 5 mL/L Bug Oil | 60 | effect seen |
| | | | 8 mL/L Bug Oil | 50 | |
| | | | 10 mL/L Bug Oil | 70 | |

Table III further illustrates the synergistic effects of combinations of BUGOIL® with abamectin, and BUGOIL® with spinosad against T. urticae in the present studies. For the combination of BUGOIL® with abamectin, the observed mean mortality was 64%, which is significantly higher than the calculated potential mean mortality of only 24% if the effects were merely additive. For the combination of BUGOIL® with spinosad, the observed mean mortality was 84%, which is significantly higher than the calculated potential mean mortality of only 24% if the effects were merely additive.

TABLE III
Synergistic Effects of Pesticidal Mixtures on the Red Spider Mite,
Tetranychus urticae

| | | |
|---|---|---|
| | Observed | Calculated potential |
| | mean % mortality | mean % mortality of T urticae |
| Treatment | of T urticae | if treatment effects additive |

Abamectin

| | | |
|---|---|---|
| 5 mL BUGOIL ®/L | 14 | |
| 0.25 g abamectin/L | 10 | |
| 5 mL BUGOIL ®/L + | 64 | 24 |
| 0.25 g abamectin/L | | |

Spinosad

| | | |
|---|---|---|
| 5 mL BUGOIL ®/L | 16 | |
| 10 g spinosad/L | 8 | |
| 5 mL BUGOIL ®/L + | 84 | 24 |
| 10 g spinsad/L | | |

### Example 13

### Summary of Effects of Pesticidal Mixtures on the Green Peach Aphid, Myzus persicae

Table IV below summarizes results of studies described in Examples 6-10 above, designed to explore the effects of varying rates of each formulation in mixtures (at rates from LD₂₅ and LD₅₀) with a fixed rate of BUGOIL®, and the effects of varying rates of BUGOIL® in mixtures (at rates from LD₂₅ and LD₅₀) with a fixed rate of each formulation, on the mortality (%) of M. persicae on infested test arenas at 72 hours after application. The mortality rates of mixtures were measured and compared with the theoretical (predicted) mortality rate of the mixture based on addition of the effects of each component of the mixture, where the percentage values reported in Table IV were rounded to the next integer.

TABLE IV
The Green Peach Aphid (M. persicae) versus Mixed Products

| Mixture | | | | |
|---|---|---|---|---|
| Bioassay | | | Concentration | |
| Study | Product/Active | Target | Ranges tested | Comments |
| CEMS-4281 | Abamectin (Vertan) | M. persicae | 10 mL/L Bug Oil + | Data inconclusive as |
| | | | 5 g a.i./ha | to any increased |
| | | | 10 g a.i./ha | effect, but good |
| | | | 15 g a.i./ha | mortality rates |
| CEMS-4281 | Abamectin (Vertan) | M. persicae | 5 g a.i./ha + | No clear improvement |
| | | | 10 mL/L Bug Oil | with increase in Bug |
| | | | 20 mL/L Bug Oil | Oil rate |
| | | | 50 mL/L Bug Oil | |
| CEMS-4283 | Imidacloprid (Confidor 20 | M. persicae | 10 mL/L Bug Oil + | Good mortality at all |
| | LS) | | 0.1 g a.i./ha | rates of imidacloprid |
| | | | 0.25 g a.i./ha | |
| | | | 0.5 g a.i./ha | |
| CEMS-4283 | Imidacloprid (Confidor 20 | M. persicae | 0.1 g a.i./ha + | No beneficial effect |
| | LS) | | 10 mL/L Bug Oil | |
| | | | 20 mL/L Bug Oil | |
| | | | 50 mL/L Bug Oil | |
| CEMS-4284 | Indoxacarb (Steward) | M. persicae | 10 mL/L Bug Oil + | High rates of |
| | | | 100 g a.i./ha | indoxacarb needed to |
| | | | 200 g a.i./ha | produce this effect |
| | | | 400 g a.i./ha | |
| CEMS-4284 | Indoxacarb (Steward) | M. persicae | 100 g a.i./ha + | As above |
| | | | 10 mL/L Bug Oil | |
| | | | 20 mL/L Bug Oil | |
| | | | 50 mL/L Bug Oil | |
| CEMS-4285 | Lambda-cyhalothrin | M. persicae | 10 mL/L Bug Oil + | Good mortality |
| | (Karate King) | | 0.01 g a.i./ha | |
| | | | 0.05 g a.i./ha | |
| | | | 0.1 g a.i./ha | |
| CEMS-4285 | Lambda-cyhalothrin | M. persicae | 0.1 g a.i./ha + | No specific |
| | (Karate King) | | 10 mL/L Bug Oil | enhancement of |
| | | | 20 mL/L Bug Oil | activity |
| | | | 50 mL/L Bug Oil | |
| CEMS-4286 | Spinosad (SpinTor) | M. persicae | 10 mL/L Bug Oil + | Variable response |
| | | | 1 g a.i./ha | |
| | | | 5 g a.i./ha | |
| | | | 10 g a.i./ha | |
| CEMS-4286 | Spinosad (SpinTor) | M. persicae | 1 g a.i./ha + | Variable and |
| | | | 10 mL/L Bug Oil | inconclusive |
| | | | 20 mL/L Bug Oil | |
| | | | 50 mL/L Bug Oil | |

### Example 14

### Field Studies of the Efficacy of BUGOIL® and Selected Acaricides/Insecticides For Controlling Adult Whiteflies on Cucurbitaceae

Field studies as described below were carried out to determine the efficacy of BUGOIL® alone, lambda cyhalothrin alone, imidacloprid alone, abamectin alone, and mixtures of BUGOIL® with lambda cyhalothrin, imidacloprid, or abamectin, for control of adult whiteflies of different whitefly species, on zucchini and cucumber. In the following studies, the dose (N) is the label dose for each product, expressed in concentration (grams active ingredient) per liter of spray mixture (a.i.)/1).

Activity of BUGOIL® and Lambda Cyhalothrin Against Adult Trialeurodes sp. on Zucchini.

Plants of the zucchini cultivar “Dedida” (Cucurbita pepo L., cv Dedida) infested with the whitefly Trialeurodes sp., were treated with BUGOIL® and/or lambda cyhalothrin (formulated as KARATE KING® 2.5 WG, active ingredient content lambda-cyhalothrin 2.5%, Syngenta Crop Protection, Batch No. SOL7E10, July 2007), where the dose prior to dilution=N=label dose=0.02 g (a.i.)/l, and the percentage control (% control) of whitefly compared to untreated whitefly-infested plants, was determined at 1 day after application (1 DAA), 3 DAA, 7, DAA, and 14 DAA for each treatment. The following treatments were applied and evaluated: BUGOIL® at 5 mL/L; BUGOIL® at 10 mL/L; lambda-cyhalothrin at N dose (lambda-cyhalothrin 2.5%); lambda cyhalothrin at N/2 dilution (1.25% lambda-cyhalothrin); BUGOIL® 5 mL/L plus lambda cyhalothrin at N/2 dilution (1.25% lambda-cyhalothrin); BUGOIL® 5 mL/L plus lambda cyhalothrin at N/6 dilution (0.8% lambda-cyhalothrin); BUGOIL® 5 mL/L plus lambda cyhalothrin at N/20 dilution (0.125% lambda-cyhalothrin). FIG. 11 presents the results as bar graphs showing the % control of whitefly at 1 DAA, 3 DAA, 7 DAA, and 14 DAA, for: BUGOIL® at 5 mL/L (BUGOIL 5), BUGOIL® at 10 mL/L (BUGOIL 10); lambda cyhalothrin (Lambda-N), lambda cyhalothrin at N/2 dilution (Lambda N/2); BUGOIL® 5 mL/L plus lambda cyhalothrin at N/2 dilution (BUGOIL 5+Lambda N/2); BUGOIL® 5 mL/L plus lambda cyhalothrin at N/6 dilution (BUGOIL 5+Lamdba N/6); and BUGOIL® 5 mL/L and lambda cyhalothrin at N/20 dilution (BUGOIL 5+Lambda N/20).

As shown in FIG. 11, BUGOIL® alone, and lambda cyhalothrin (as KARATE KING®2.5 WG) alone, provided control of adult whiteflies in excess of 60%, at the rates tested in this experiment. Little or no additive effects was observed for mixtures of BUGOIL® (at 5 mL/L) and lambda cyhalothrin at different rates, up to 14 days after application (14 DAA).

Activity of BUGOIL® and Imidacloprid Against Adult Trialeurodes Sp. on Zucchini

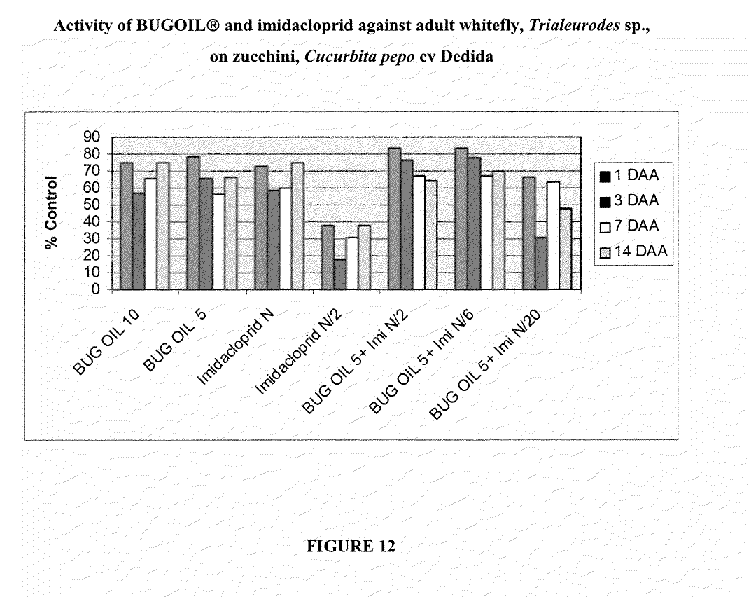

Plants of the zucchini cultivar “Dedida” (Cucurbita pepo L., cv Dedida) infested with the whitefly Trialeurodes sp., were treated with BUGOIL® and/or imidacloprid (formulated as CONFIDOR® 20 LS, active ingredient content 20% imidacloprid, Bayer Crop Science, Spain, Batch No. EQ6000353/5, November 2007), where the dose prior to dilution=N=label dose=0.15 g (a.i.)/l, and the percentage control (% control) of whitefly compared to untreated whitefly-infested plants, was determined at 1 day after application (1 DAA), 3 DAA, 7, DAA, and 14 DAA for each treatment. The following treatments were applied and evaluated: BUGOIL® at 5 mL/L; BUGOIL® at 10 mL/L; imidacloprid at N dose; imidacloprid at N/2 dilution; BUGOIL® 5 mL/L plus imidacloprid at N/2 dilution; BUGOIL® 5 mL/L plus imidacloprid at N/6 dilution; BUGOIL® 5 mL/L plus imidacloprid at N/20 dilution. FIG. 12 presents the results as bar graphs showing the % control of whitefly at 1 DAA, 3 DAA, 7 DAA, and 14 DAA, for: BUGOIL® at 5 mL/L (BUGOIL 5); BUGOIL® at 10 mL/L (BUGOIL 10); imidacloprid (Imidacloprid N); imidacloprid at N/2 dilution (Imidacloprid N/2); BUGOIL® 5 mL/L plus imidacloprid at N/2 dilution (BUGOIL 5+Imi N/2); BUGOIL® 5 mL/L plus imidacloprid at N/6 dilution (BUGOIL 5+Imi N/6); and BUGOIL® 5 mL/L plus imidacloprid at N/20 dilution (BUGOIL 5+Imi N/20).

As shown in FIG. 12, BUGOIL® alone at 5 mL/L and 10 mL/l, and imidacloprid as CONFIDOR® 20 LS (20% SC) alone at the usual application rate (N), provided control of adult whiteflies in excess of 70-80%. Imidacloprid alone, at the N/2 dilution of the usual application rate, provided control of adult whiteflies, but at rates of only 30-40%. Combinations of BUGOIL® with all concentrations of imidacloprid as CONFIDOR® 20 LS (20% SC) (i.e., at N/2 dilution, N/6 dilution, and N/20 dilution), consistently gave better control than either product alone, at 1 DAA, 3 DAA, and 7 DAA.

Activity of BUGOIL® and Abamectin Against Bemisia tabaci on Cucumber

Cucumber (Cucumis sativus) infested with Bemisia tabaci were treated with BUGOIL® and/or abamectin (formulated as VERTAN® 1.8 EC (1.8% EC), active ingredient content 1.8% abamectin (formulated as 80% abamectin B₁ₐ and 20% B_{1b}), Laboratorios Alcotán S.A., Dos Hermanas (Sevilla), Spain, Batch No. 10803007, April 2008), where the dose prior to dilution=N=label dose=0.0.18 g (a.i.)/1, and the percentage control (% control) of whitefly compared to untreated whitefly-infested plants, was determined at 1 day after application (1 DAA), 3 DAA, 7, DAA, and 14 DAA for each treatment. The following treatments were applied and evaluated: BUGOIL® at 5 mL/L; BUGOIL® at 10 mL/L; abamectin at N dose; abamectin at N/2 dilution; BUGOIL® 5 mL/L plus abamectin at N/2 dilution; BUGOIL® 5 mL/L plus abamectin at N/6 dilution; BUGOIL® 5 mL/L plus abamectin at N/20 dilution. FIG. 13 presents the results as bar graphs showing the % control of whitefly at 1 DAA, 4 DAA, 7 DAA, and 14 DAA, for: BUGOIL® at 5 mL/L (BUGOIL 5); BUGOIL® at 10 mL/L (BUGOIL 10); abamectin (Abamectin N); abamectin at N/2 dilution (Abamectin N/2); BUGOIL® 5 mL/L plus abamectin at N/2 dilution (BUGOIL 5+Aba N/2); BUGOIL® 5 mL/L plus abamectin at N/6 dilution (BUGOIL 5+Aba N/6); BUGOIL® 5 mL/L plus abamectin at N/20 dilution (BUGOIL 5+Aba N/20).

As shown in FIG. 13, BUGOIL® alone at 5 mL/L and 10 mL/L, and abamectin as VERTAN® 1.8 EC (1.8% EC) alone at the usual application rate (N) and at N/2 dilution, provided control of adult Bemisia tabaci whiteflies in excess of 60% at 1 DAA, and over 80& at 4DAA and 7 DAA. 70-80%. BUGOIL® 5 mL/L plus abamectin at N/2 dilution gave the highest control rate at all assessment times, providing control in excess of 80% at 1 DAA, 4 DAA, 7 DAA, and 14 DAA. No major improvements in activity were observed using mixtures of BUGOIL® and abamectin as VERTAN® 1.8 EC (1.8% EC). However, at 14 DAA the level of control is higher for the mixtures than for single component treatments, indicating that mixing BUGOIL® with abamectin (specifically, abamectin as VERTAN® 1.8 EC (1.8% EC)) can provide improved persistence of control of adult whiteflies on cucumber.

### Example 15

### Field Studies of the Efficacy of BUGOIL® and Selected Acaricides/Insecticides For Controlling Aphids and Whitefly Nymphs on Cotton

Field studies as described below were carried out to determine the efficacy of BUGOIL® alone, lambda cyhalothrin alone, imidacloprid alone, abamectin alone, and mixtures of BUGOIL® with lambda cyhalothrin, imidacloprid, or abamectin, for control of whitefly nymphs on cotton, and for control of aphids on cotton.

Lambda cyhalothrin is formulated as WARRIOR® (WARRIOR® with Zeon Technology, active ingredient content 11.4% lambda cyhalothrin, capsule suspension, Syngenta Crop Protection) where the dose prior to dilution=N=label dose=5.12 fl. oz./acre.

Imidacloprid is formulated as PROVADO® 1.6 F (PROVADO® 1.6 Flowable, active ingredient content 17.4% 1-[(6-chloro-3-pyridinyl)methyl]-N-nitro-2-imidazolidinimine, flowable insecticide, Bayer Corporation Crop Protection Products) where the dose prior to dilution=N=label dose=5 fl. oz./acre.

Abamectin is formulated as ZEPHYR® 0.15 EC (ZEPHYR® 1.5 Emulsified Concentrate, active ingredient content 2.0% abamectin (80% abamectin B₁ₐ and 20% B_{1b}), Ivorychem Pte.) where the dose prior to dilution=N=label dose=16 fl. oz./acre.

Whitefly nymphs on cotton. Cotton plants were treated with BUGOIL® alone, lambda cyhalothrin alone, imidacloprid alone, abamectin alone, and mixtures of BUGOIL® with lambda cyhalothrin, imidacloprid, or abamectin, and effects on control of whitefly nymphal stages were measured after application.

BUGOIL® alone, lambda cyhalothrin alone, imidacloprid alone, abamectin alone, and mixtures of BUGOIL® with lambda cyhalothrin, imidacloprid, or abamectin, all provided approximately 50-70% control of whitefly nymphal stages. Limited improvements in efficacy were seen with mixtures of BUGOIL® with lambda cyhalothrin, imidacloprid, or abamectin.

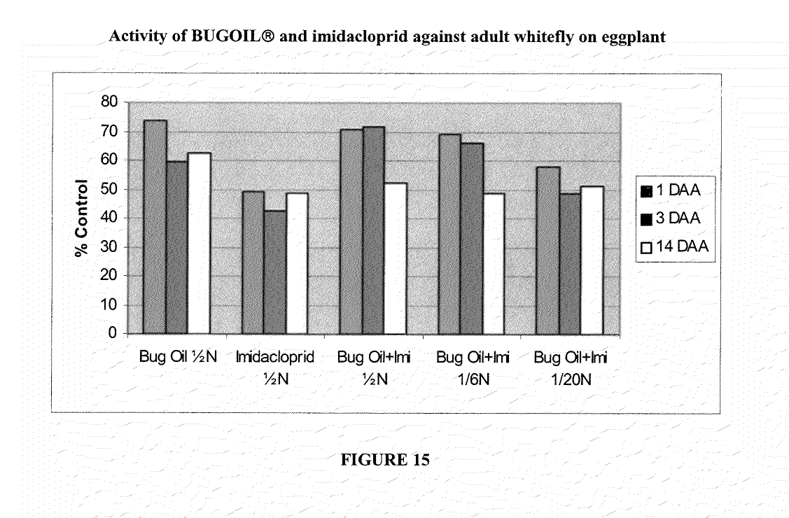

FIG. 15 shows that at 22 days after application (22 DAA), mixtures of BUGOIL® at 5 mL/L with various concentrations of lambda cyhalothrin (FIG. 15A), or BUGOIL® at 5 mL/L with various concentrations of abamectin (FIG. 15B), gave better control than either component used alone.

FIG. 15A shows that the mixture of BUGOIL® at 5 mL/L with lambda cyhalothrin at N/2 dilution (BUGOIL+Lam ½N) and the mixture of BUGOIL® at 5 mL/L with lambda cyhalothrin at N/20 dilution (BUGOIL+Lam 1/20N) provide a higher rate of control at 22 DAA than BUGOIL® alone, or lambda cyhalothrin alone, at a lower concentration of lambda cyhalothrin.

FIG. 15B shows that the mixture of BUGOIL® at 5 mL/L with abamectin at N/2 dilution (BUGOIL+Aba ½N) provides a higher rate of control at 22 DAA than BUGOIL® alone, or abamectin alone. FIG. 15B shows that the mixtures of BUGOIL® at 5 mL/L with abamectin at N/6 dilution (BUGOIL+Aba ⅙N), or BUGOIL® at 5 mL/L with abamectin at N/20 dilution (BUGOIL+Aba 1/20N), provide a higher rate of control than BUGOIL® alone at either concentration, and provide an equivalent or a higher rate of control than higher concentrations of abamectin alone. The results for mixtures of BUGOIL® with abamectin indicate that these mixtures can provide improved persistence of control of whitefly nymphs on cotton.

Aphids on cotton. In the field trial, only low to moderate levels of aphids, Aphis gossypii, were present on the cotton plants. No reliable trends for aphid control were observed when BUGOIL®, alone or in mixtures with lambda cyhalothrin, abamectin, or imidacloprid.

### Example 16

### Field Studies of the Efficacy of BUGOIL® and Selected Acaricides/Insecticides For Controlling Whitefly, Leafhoppers, Aphids, and Lepidoptera on Eggplant

Field studies as described below were carried out to determine the efficacy of BUGOIL®, lambda cyhalothrin, imidacloprid, and abamectin, alone and in mixtures with BUGOIL®, for control of the whitefly Bemisia tabaci, the leafhopper Empoasca biggutula, the aphid Aphis gosspyii, the shoot/fruit borer Leucinodes orbonalis and the cutworm Spodoptera litura on the eggplant cultivar “Casino” (Solanum melongena cv Casino). In the following studies, the dose (N) is the label dose for each product, expressed in concentration (grams active ingredient) per liter of spray mixture (a.i.)/l).

Lambda cyhalothrin is formulated as Karate® (KARATE® with Zeon Technology, Syngenta Phillipines, Inc., Batch No. JAK8,18179, Production Date Nov. 11, 2008, Phillipines Department of Agriculture, Fertilizer and Pesticides Authority (FPA) Reg. No. 011-204-0396), where the dose prior to dilution=N=label dose=0.8 g (a.i.)/l (rated adapted to lepidopterian and leafhopper pests).

Imidacloprid is formulated as CLIMAX® (CLIMAX® 200 SL, Bayer CropScience, Inc., Batch No. 81001025, Production Date Feb. 22, 2005, FPA Reg. No. 284-240-0761 where the dose prior to dilution=N=label dose=0.15 g (a.i.)/l.

Abamectin is formulated as AGRI-MEK® (AGRI-MEK® 1.8 EC, Syngenta Phillipines, Inc., FPA Reg. No. 011-234-0791), where the dose prior to dilution=N=label dose=0.018 g (a.i.)/l.

BUGOIL® at both 5 and 10 mL/L provided fair to good control of sucking insect pests (whitefly, leafhoppers and aphids) and good to very good control of lepidopteran pests (cutworm and borer). Abamectin (as AGRI-MEK® 1.8 EC) also showed good effects, but improvements were achieved when reduced rates of BUGOIL® and abamectin were applied in mixtures, both in terms of improved control and increased persistence of effect.

An experiment was conducted to test the efficacy of BUGOIL® and/or abamectin (as AGRI-MEK® 1.8 EC) for control of the leafhopper E. biggutula. At 3 days after the first application (3 days after application 1), BUGOIL® alone at 5 mL/L gave 88% control, abamectin along gave 78% control and a mixture of BUGOIL® and abamectin gave 89% control. At 3 days after the second application (3 days after application 2), BUGOIL® alone at 5 mL/L gave 51% control, abamectin alone gave 66% control, and a mixture of BUGOIL® and abamectin gave 85% control. At 3 days after the third application (3 days after application 3), BUGOIL® alone at 5 mL/L gave 56% control, abamectin alone gave 49% control and a mixture of BUGOIL® and abamectin gave 72% control.

An experiment was conducted to test the efficacy of BUGOIL® and imidacloprid (as CLIMAX® 200 SL) for control of adult whitefly, B. tabaci. As shown in FIG. 15, over a 14 day test period, BUGOIL® alone provided over 60% control of adult whitefly, imidacloprid alone gave 50% control or less. In mixture, little benefit was gained with the exception that the effectiveness of the mixture was higher at 3 days after application (3DAA).

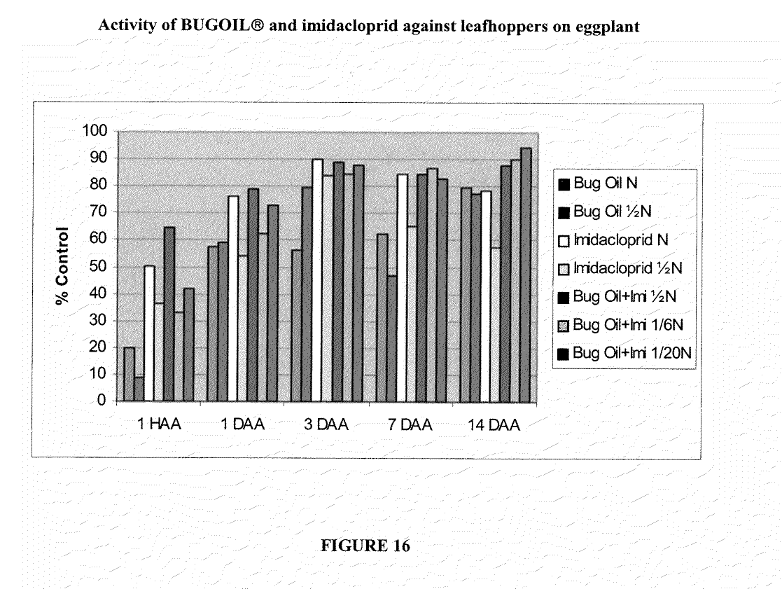

An experiment was conducted to test the efficacy of BUGOIL® and imidacloprid (as CLIMAX® 200 SL) for control of the leafhopper E biggutula. As shown in FIG. 16, there was some indication that the effectiveness of a mixture of BUGOIL® with imidacloprid was numerically better than the effectiveness of each individual components of the mixture when used alone. As shown in FIG. 16, at 3 days after application (3DAA), a half rate of BUGOIL® alone (BUGOIL® ½ N, 5 mL/L, BUGOIL®) imidacloprid alone, and all mixtures of BUGOIL® with imidacloprid gave 80-90% control of leafhoppers. The speed of effect should be noted: at 1 hour after application (1 HAA), BUGOIL® alone at the normal rate (BUGOIL® N, 10 mL/L BUGOIL®) controlled about 20% of leafhoppers, a half rate of BUGOIL® alone (BUGOIL® ½ N, 5 mL/L, BUGOIL®) controlled less than 10% of leafhoppers, and a half rate of imidacloprid alone at half the normal rate (Imidacloprid ½ N) gave 37% control, whereas a mixture of BUGOIL® at the normal rate and imidacloprid at half the normal rate (BUGOIL+Imi ½N) gave 65% control.

An experiment was conducted to test the efficacy of BUGOIL® and imidacloprid (as CLIMAX® 200 SL) for control of the leafhopper E biggutula after a single application. All treatments, i.e., BUGOIL® alone, abamectin alone, and mixtures of BUGOIL® and abamectin gave around 80% control at 3 days after application.

An experiment was conducted to test the efficacy of BUGOIL®, lambda cyhalothrin (as KARATE®), abamectin (as AGRI-MEK® 1.8 EC) and imidacloprid (as CLIMAX® 200 SL) against the cutworm, S litura. At 7 days after application (7 DAA), the half rate of BUGOIL® alone (BUGOIL® ½ N, 5 mL/L, BUGOIL®) always gave 50% control or better, while imidacloprid alone and lambda cyhalothrin alone gave around 60% control at the recommended application rate (N), and 40-50% control at half the recommended rate (½ N). Abamectin alone gave 70% control, but no real improvements were observed with any of the mixtures against cutworms.

Thus, field experiments to determine the efficacy of mixtures of BUGOIL® with three acaricides/insecticides (lambda cyhalothrin, imidacloprid, abamectin) against whiteflies, leafhoppers, aphids, borers and cutworms on eggplant showed some benefit from using BUGOIL® in mixture with abamectin, and certain showed improved speed of effects, where better control of whitefly with mixtures of BUGOIL® plus imidacloprid was seen by 3 days after application (3DA), and better control of leafhoppers with mixtures of BUGOIL® plus imidacloprid was apparent after 1 hour (1HAA).

Various modifications can be made to the preferred embodiments without departing from the spirit and scope of the invention as defined in the appended claims.

What is claimed is:

1. A synergistic pesticidal mixture comprising a first pesticidal composition comprising at least two essential oils, a carrier oil, and an emulsifier, and a second pesticidal composition comprising at least one insecticide, in a synergistically effective amount wherein the pesticidal activity of the mixture against a target pest is greater than the sum of the activity of the first pesticidal composition alone and the activity of the second pesticidal composition alone.

2. The synergistic pesticidal mixture of claim 1, wherein the first pestidical composition comprises tagetes oil and a thymol-containing oil.

3. The synergistic pesticidal mixture of claim 2, wherein the thymol-containing oil is thyme oil.

4. The synergistic pesticidal mixture of claim 3, comprising tagetes oil at about 0.600% w/w, thyme oil at about 0.600% w/w, canola oil at about 93.799% w/w, Tween20® at about 5.000% w/w, and wintergreen oil at about 0.0001% w/w.

5. The synergistic pesticidal mixture of claim 1, wherein the second pestidical composition comprises at least one insecticide selected from the group consisting of abamectin, imidacloprid, indoxacarb, lambda-cyhalothrin, and spinosad.

6. The synergistic pesticidal mixture of claim 1, wherein the target pest is an arthropod pest or an insect pest.

7. The synergistic pesticidal mixture of claim 6, wherein the target pest is an arthropod pest.

8. The synergistic pesticidal mixture of claim 7, wherein the arthropod pest is a mite.

9. The synergistic pesticidal mixture of claim 8, wherein the first pesticidal composition comprises a mixture of tagetes oil and thyme oil, the second pesticidal composition comprises abamectin or spinosad, and the target pest is a spider mite.

10. The synergistic pesticidal mixture of claim 9, wherein the first pesticidal composition comprises a mixture of tagetes oil and thyme oil, the second pesticidal composition comprises abamectin and the target pest is the red spider mite, Tetranychus urticae.

11. The synergistic pesticidal mixture of claim 9, wherein the first pesticidal composition comprises a mixture of tagetes oil and thyme oil, the second pesticidal composition comprises spinosad, and the target pest is the red spider mite, Tetranychus urticae.

12. The synergistic pesticidal mixture of claim 6, wherein the target pest is an insect pest.

13. The synergistic pesticidal mixture of claim 12, wherein the insect pest is an aphid, a lepidopteran, or an hemipteran.

14. A method for identifying synergistic pesticidal mixtures comprising, measuring the activity against a target pest of a first pesticidal composition comprising at least two essential oils, a carrier oil, and an emulsifier, measuring the activity against the target pest of a second pesticidal composition comprising at least one insecticide, and measuring the activity against the target pest of a mixture comprising the first pesticidal composition in an amount having known activity, and the second pesticidal composition in an amount having known activity, wherein a mixture having higher activity against the target pest than the sum of the activity of the first pesticidal composition alone and the activity of the second pesticidal composition alone is identified as a synergistic pesticidal mixture.
